(19) United States
(12) Patent Application Publication
(10) Pub. No.: US 2025/0288600 A1
(43) Pub. Date: Sep. 18, 2025

(54) USES OF FARNESOID X RECEPTOR AGONISTS

(71) Applicant: Intercept Pharmaceuticals, Inc., Morristown, NJ (US)

(72) Inventor: Thomas CAPOZZA, Barrington, RI (US)

(21) Appl. No.: 18/858,674

(22) PCT Filed: Apr. 21, 2023
(86) PCT No.: PCT/US2023/019448

§ 371 Date: Oct. 21, 2024

Related U.S. Application Data
(60) Provisional application No. 63/333,287, filed on Apr. 21, 2022.
(60) Provisional application No. 63/381,752, filed on Oct. 31, 2022.

(51) Int. Cl. A61K 31/575; A61K 45/06; A61P 1/16

(52) U.S. Cl. CPC A61K 31/575; A61K 45/06; A61P 1/16

(57) ABSTRACT

The present invention relates to FXR agonist compounds and methods of use thereof for treating, ameliorating, or promoting recovery from diseases and disorders, such as acute diseases of the liver, for example, severe alcohol-associated hepatitis.

### CROSS REFERENCE TO RELATED APPLICATIONS

This application claims priority benefit of U.S. Provisional Application No. 63/333,287 filed Apr. 21, 2022, and U.S. Provisional Application No. 63/381,752 filed Oct. 31, 2022, the contents of each of which are incorporated herein by reference in their entireties.

### TECHNICAL FIELD

The present invention relates to compounds and methods of use thereof for treating, ameliorating, or promoting recovery from diseases and disorders, such as acute diseases of the liver.

### BACKGROUND OF THE INVENTION

Alcoholic hepatitis (also known as alcohol-associated hepatitis or acute alcohol-associated hepatitis) is an acute form of alcohol-induced liver injury that occurs with the consumption of a large quantity of alcohol over a prolonged period. Alcoholic hepatitis can range in severity from asymptomatic derangement of biochemistries to liver failure and death. Severe alcoholic hepatitis is a severe form of an acute alcoholic liver disease caused by excessive alcohol consumption which may be characterized by rapid onset of jaundice, malaise, tender hepatomegaly, and features of systemic inflammatory response (Shah N.J., Royer A, John S. Alcoholic Hepatitis. Stat Pearls. 2021 January; 1-8; hereby incorporated by reference). Severe alcoholic hepatitis (sAH) is a life-threatening medical condition with an average 30-day mortality as high as 17% to 50%, and an average 1-year mortality of about 56% (Mathurin P., et al. Gastroenterology 1996 June; 110(6):1847-53; Thursz, M. R., et al. N Engl J Med. 2015 Apr. 23; 372(17):1619-28; hereby incorporated by reference). Treatment options for patients with sAH are limited to systemic corticosteroids (unless there are contraindications such as sepsis, infection, gastrointestinal bleeding or acute kidney injury) along with supportive care, nutrition, and alcohol abstinence. Corticosteroids do not impart long-term benefit in patients with sAH and their use is limited due to the risk of infection and GI bleeding. Further, liver transplantation for alcohol-related liver disease is currently at an all-time high (Noureddin, N., et al. Transplant Direct 2020 Oct. 8; 6(11):e612), and the number of patients in the US awaiting and undergoing liver transplantation for acute alcohol-associated hepatitis has increased substantially during the COVID-19 pandemic (Bitterman, T., et al. JAMA Netw. Open. 2021; 4(7):e2118713). Unfortunately, patients with sAH and progressive liver failure who are non-responders to corticosteroid therapy and ineligible for liver transplantation are frequently referred for palliative care (Singal, A. K., et al. Am J Gastroenterol. 2018; 113(2):175-94; hereby incorporated by reference).

Another example of an acute liver disease with insufficient treatment options is acute decompensated liver disease, also known as acute-on-chronic liver failure (ACLF). ACLF is a syndrome characterized by acute decompensation of chronic liver disease associated with organ failures and high short-term mortality. Alcoholic hepatitis and chronic viral hepatitis are the most common underlying liver diseases. Up to 40%-50% of the cases of ACLF have no identifiable trigger; in the remaining patients, sepsis, active alcoholism, and relapse of chronic viral hepatitis are the most common reported precipitating factors. An excessive systemic inflammatory response seems to play a crucial role in the development of ACLF.

Thus, there remains a need for additional therapies for treating acute liver diseases such as sAH and ACLF. The present invention addresses this need and provides other related advantages.

### SUMMARY OF THE INVENTION

In one aspect, the present invention provides a method of treating an acute liver disease comprising administering to a patient in need thereof an effective amount of Compound 1:

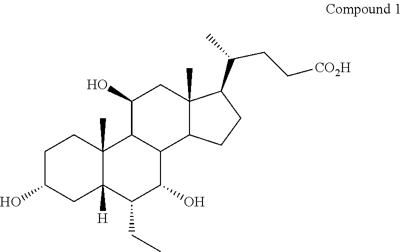 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt or amino acid conjugate thereof.

In one aspect, the present invention provides a method of treating an acute liver disease comprising administering to a patient in need thereof an effective amount of Compound 1 or a pharmaceutically acceptable salt or amino acid conjugate thereof, wherein the acute liver disease is alcoholic hepatitis (AH).

In another aspect, the present invention provides a method of treating an acute liver disease comprising administering to a patient in need thereof an effective amount of Compound 1 or a pharmaceutically acceptable salt or amino acid conjugate thereof, wherein the acute liver disease is severe alcoholic hepatitis (sAH).

In yet another aspect, the present invention provides a method of treating an acute liver disease comprising administering to a patient in need thereof an effective amount of Compound 1 or a pharmaceutically acceptable salt or amino acid conjugate thereof, wherein the acute liver disease is acute-on-chronic liver failure (ACLF).

In one aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a Maddrey discriminant function (MDF) score of ≥32 prior to treatment with Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a model for end stage liver disease (MELD or MELD-Na) score of ≥18, or ≥20, or ≥21 prior to treatment with Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a MDF score of 32 to 60.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a MELD score of 18, or 20 or 21 to 25 or 30 or MELD-Na score of 18, or 20 or 21 to 25 or 30.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a MELD score of 21 to 30 prior to treatment with Compound 1 or a pharmaceutically acceptable salt or amino acid conjugate thereof.

In one aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits a Maddrey discriminant function (MDF) score of ≥32, and a MELD score of 21 to 30 (inclusive) prior to treatment with Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits an AST of ≥ (greater than or equal to) 50 U/L (or IU/L) prior to treatment with Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits an AST/ALT ratio of ≥1.5 prior to treatment with Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits prior to treatment with Compound 1 or a pharmaceutically acceptable salt or amino acid conjugate thereof one or more of an AST of ≥50 U/L; an AST/ALT ratio of ≥1.5; a Maddrey discriminant function (MDF) score of ≥32; and/or a model for end-stage liver disease (MELD or MELD-Na) score of ≥18 or ≥20 or ≥21, for example from 21-30.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method reduces the MELD or MELD-Na score of the patient by at least 3 and/or reduces the MDF score of the patient by at least 3 by day 28 of treatment.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 1.0 mg to about 300 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 2.5 mg to about 300 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound us administered daily at a dose of from about 5 mg to about 120 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 is administered daily at a dose of from about mg to about 100 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 is administered daily at a dose of from about mg to about 50 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 is administered daily at a dose of from about 5 mg to about 25 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 is administered daily at a dose of from about 5 mg to about 10 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 5 mg to about 150 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 5 mg to about 120 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 10 mg to about 100 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered daily at a dose of about 50 mg to about 250 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 is administered daily at a dose of about 2.5 mg, about 5 mg, about 10 mg, about 15 mg, about 20 mg, about 25 mg, about 35 mg, about 45 mg, about 50 mg, about 60 mg, about 75 mg, about 85 mg, about 100 mg, about 115 mg, about 120 mg, about 125 mg, about 140 mg, about 150 mg, about 165 mg, about 175 mg, about 190 mg, or about 200 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered in single or multiple doses sufficient to achieve a total daily dose of about 1.0 mg to about 300 mg; 1.0 mg to about 300 mg; or about 2.5 mg to about 300 mg; or about 5 mg to about 100 mg; or about 5 mg to about 50 mg; or about 5 mg to about 25 mg; or about 5 mg to about 10 mg; or about 5 mg to about 150 mg; or about 5 mg to about 120 mg; or about 10 mg to about 100 mg; or about 50 mg to about 200 mg; or about 2.5 mg, about 5 mg, about 10 mg, about 15 mg, about 20 mg, about 25 mg, about 35 mg, about 45 mg, about 50 mg, about 60 mg, about 75 mg, about 85 mg, about 100 mg, about 115 mg, about 120 mg, 125 mg, about 140 mg, about 150 mg, about 165 mg, about 175 mg, about 190 mg, or about 200 mg.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, further comprising monitoring blood levels of a biomarker in the patient after administration of Compound 1 or a pharmaceutically acceptable salt or amino acid conjugate thereof, wherein the biomarker is selected from C4, FGF-19, and endogenous bile acids.

In another aspect, the present invention provides a method of treating an acute liver disease selected from sAH and ACLF, comprising administering to a patient in need thereof an effective amount of Compound 1 or a pharmaceutically acceptable salt or amino acid conjugate thereof, wherein the patient has a chronic liver disease in addition to sAH or ACLF.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient has alcohol-related (or alcohol-associated) liver disease (ALD), previously known as alcoholic liver disease.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits one or more of the following: (1) ongoing alcohol consumption of more than 40 g of alcohol per day if the patient is a woman and more than 60 g of alcohol per day if the patient is a man for 6 months or more, with less than 60 days of abstinence before the onset of jaundice; (2) the presence of elevated liver enzymes (aspartate aminotransferase [AST] and alanine aminotransferase [ALT] concentrations≥50 IU/L but ≤400 IU/L and an AST/ALT ratio of −1:5); and (3) worsening jaundice, with bilirubin concentrations greater than 3 mg/dL.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient exhibits alcohol-associated cirrhosis, liver fibrosis, steatosis, steatohepatitis, alcoholic hepatitis, or intestinal dysbiosis.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient has previously been diagnosed with sAH and has suffered at least one relapse of sAH.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves 30-day survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves 60-day survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves 90-day survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves 90-day transplant-free survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves 28-day survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves the 6-month survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves the 6-month transplant-free survival of the patient versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves the patient's MELD and/or Lille scores at day 28 and/or day 90 after administration of Compound 1 or a pharmaceutically acceptable salt or amino acid conjugate thereof, versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves the patient's occurrence of hospital re-admission for alcohol-associated hepatitis versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves one or more of: the patient's length of stay in the hospital, the number of days the patient spends in the ICU, the number of major medical procedures performed on the patient, and/or the number of emergency room visits by the patient, versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves one or more pharmacokinetic and pharmacodynamic biomarkers, including: change from Baseline in one or more serum liver biochemistries selected from ALP, AST, ALT, GGT, and total and direct bilirubin; change from Baseline in IL-6, hs-CRP, CK-18, and TNF-α at Day 28; change from Baseline in Lipoproteins (LDL, HDL, VLDL), total cholesterol, and/or triglycerides at Day 28; and change from Baseline in lipid metabolism (LBP), 16S rDNA and/or stool sample for alpha-1-antitrypsin and microbiome/metabolome analysis; versus an otherwise similar patient who was not administered Compound 1 or a pharmaceutically acceptable salt or amino acid conjugate thereof.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves survival by about 10% to about 40% (relative to the standard of care (SOC)).

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves survival by about 10% to about 30%.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method improves survival by about 10% to about 20%.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method provides a reduction in health care utilization.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method provides a reduction in liver-related events in patients with sAH.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method provides a decrease in the patient's health care utilization by at least 10% for 60 days.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method reduces one or more of hospital re-admission within 30-days after discharge, number of hospitalizations, length of stay, emergency room visits, intensive care unit (ICU) days, infectious complications selected from sepsis, pneumonia, urinary tract infection (UTI), cellulitis, spontaneous and bacterial peritonitis (SBP), and major medical procedures.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, further comprises co-administering to the patient an effective amount of anti-inflammatory agent, an antibiotic, a probiotic, a fecal transplant, Zn-supplement or a combination of any of those agents.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, further comprises co-administering to the patient an effective amount of pentoxifylline, Augmentin, bovine colostrum, corticosteroid (e.g., prednisolone or methylprednisolone), an ELAD agent, canakinumab, a TNF inhibitor, IL-22, N-acetylcysteine, metadoxine, IgG anti-LPS, a probiotic, a fecal transplant, Zn-supplement, or a combination of any of those agents.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the patient does not exhibit one or more of: AST or ALT>400 U/L, MDF>60 prior to treatment, MELD score>25 or >30 or MELD-Na>25 or >30 prior to treatment, other causes of liver disease selected from chronic hepatitis B (hepatitis B surface antigen [HBsAg] positive), chronic hepatitis C (HCV RNA positive), acetaminophen hepatotoxicity, biliary obstruction, and autoimmune liver disease; concomitant of previous history of hepatocellular carcinoma (HCC), a history of liver transplantation, untreated sepsis, known positivity for human immunodeficiency virus infection, uncontrolled gastrointestinal (GI) bleeding or controlled GI bleeding within 7 days of beginning treatment that was associated with shock or required transfusion of more than 3 units of blood, acute kidney injury defined as a serum creatinine>133 μmol/L (>1.5 mg/dL) or the requirement for renal replacement therapy, portal vein thrombosis, acute pancreatitis, or severe associated disease selected from cardiac failure, acute myocardial infarction, severe cardiac arrhythmias, severe pulmonary disease, and neurologic disease.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method provides at least a 15% reduction in mortality at 90 days compared to standard of care (SOC).

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein the method provides a reduction in hospitalization (length of stay) by at least 15% and/or a reduction in progression to transplant by at least 15%.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered to the patient for about 30 to about 90 days.

In another aspect, the present invention provides a method of treating an acute liver disease (e.g., AH, sAH or ACLF), comprising administering to a patient in need thereof an effective amount of Compound 1 or a pharmaceutically acceptable salt or amino acid conjugate thereof, wherein Compound 1 or a pharmaceutically acceptable salt or amino acid conjugate thereof is administered orally.

In another aspect, the present invention provides a method of determining candidacy of a patient for therapeutic treatment for sAH, comprising determining the patient's MELD-Na score, wherein the patient's MELD-Na score is ≥18, for example from 21-30. In some embodiments, the method further comprises determining the patient's MDF score, for example wherein the patient's MDF score is from 32 to 60 inclusive.

In another aspect, the present invention provides a method for treating sAH comprising selecting a patient having a MELD-Na score≥18, for example from 21-30; and administering a therapeutic treatment for sAH, for example wherein the patient also has a MDF score≥32, for example from 32 to 60 inclusive. In some embodiments the therapeutic treatment comprises administering a pharmacologic agent to the patient, and/or a liver transplant.

### BRIEF DESCRIPTION OF THE DRAWINGS

FIG. 1 and FIG. 2: clinical chemistry results and liver and ileum histology from mouse alcoholic liver disease (ALD) study (Compound 1 is designated as Comp 1 and Compound 2 is designated as Comp 2).

FIG. 3: Study design for a Phase 2a, Randomized, Double-Blind, Placebo Controlled, Multicenter, Dose-escalation, Proof-of-Concept Study Evaluating the Safety, Tolerability, Efficacy and Pharmacokinetics of Compound 1 (Comp 1) in Subjects with Severe Alcohol-Associated Hepatitis (sAH).

### DETAILED DESCRIPTION OF CERTAIN EMBODIMENTS OF THE INVENTION

### 1. General Description of Exemplary Embodiments

The present invention provides a method of treating an acute liver disease comprising administering to a patient in need thereof an effective amount of an FXR agonist. In one aspect, the present invention provides a method of treating alcoholic hepatitis (AH), comprising administering to a patient in need thereof an effective amount of an FXR agonist. In one aspect, the present invention provides a method of treating an acute liver disease selected from severe alcoholic hepatitis (sAH) and acute-on-chronic liver failure (ACLF), comprising administering to a patient in need thereof an effective amount of an FXR agonist.

In one aspect, the present invention provides a method of treating severe alcoholic hepatitis (sAH) comprising administering to a patient in need thereof an effective amount of an FXR agonist. The present invention provides a method of treating sAH which represent a significant advance over systemic corticosteroid therapy, the standard of care in the treatment of sAH.

The present invention further provides a method of treating ACLF comprising administering to a patient in need thereof an effective amount of an FXR agonist.

In some embodiments, the FXR agonist is

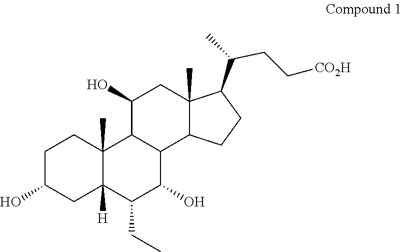 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

### 3a,7a,11β-trihydroxy-6α-ethyl-5β-cholan-24-oic acid Also Known as TC-100

or a pharmaceutically acceptable salt or amino acid conjugate thereof. In some embodiments, the FXR agonist is a tauro- or glyco-conjugate of Compound 1, or a pharmaceutically acceptable salt thereof. Compound 1 may be prepared using methods known in the art, for example, as described in U.S. Pat. Nos. 11,066,437, 11,034,717, and 9,611,289, each of which is hereby incorporated by reference in its entirety. These patents also describe various FXR agonists which may be used in accordance with the present invention.

In one aspect, the present invention provides an FXR agonist for use in treating an acute liver disease. In one aspect, the present invention provides an FXR agonist for use in treating alcoholic hepatitis (AH). In one aspect, the present invention provides an FXR agonist for use in treating an acute liver disease selected from severe alcoholic hepatitis (sAH) and acute-on-chronic liver failure (ACLF).

In another aspect, the present invention provides Compound 1, or a pharmaceutically acceptable salt or amino acid conjugate thereof, for use in treating an acute liver disease. In some embodiments, the acute liver disease is alcoholic hepatitis (AH). In some embodiments the acute liver disease is severe alcoholic hepatitis (sAH). In some embodiments the acute liver disease is moderate alcoholic hepatitis. In some embodiments the acute liver disease is mild alcoholic hepatitis. In some embodiments, the acute liver disease is acute-on-chronic liver failure (ACLF).

In one aspect, the present invention provides Compound 1, or a pharmaceutically acceptable salt or amino acid conjugate thereof, for use in treating alcoholic hepatitis (AH). In one aspect, the present invention provides Compound 1, or a pharmaceutically acceptable salt or amino acid conjugate thereof, for use in treating an acute liver disease selected from severe alcoholic hepatitis (sAH) and acute-on-chronic liver failure (ACLF).

In some embodiments, the patient has a chronic liver disease in addition to sAH or ACLF. In some embodiments, the chronic liver disease is a cholestatic liver disease. In some embodiments, the chronic liver disease is a non-cholestatic liver disease.

In some embodiments, the patient exhibits a chronic liver condition in addition to the acute liver disease, such as AH or sAH or ACLF. In some embodiments, the acute liver disease or condition is present in addition to the chronic liver disease or condition which can be selected from, but not limited to, cirrhosis (alcohol-related cirrhosis), liver fibrosis, alcohol-related steatohepatitis (ASH), steatosis, or cholestatic liver disease. In some embodiments, the acute liver disease or condition is present in addition to the chronic liver disease or condition which can be selected from, but not limited to, cirrhosis, liver fibrosis, alcohol-related steatohepatitis, or steatosis. In some embodiments, the patient exhibits cirrhosis. In some embodiments, the patient exhibits liver fibrosis. In some embodiments, the patient exhibits steatosis. In some embodiments, the patient exhibits ASH. In some embodiments, the patient exhibits alcoholic hepatitis.

In some embodiments, the patient has ALD selected from early ALD (stage 0 2) or advanced ALD (bridging fibrosis, stage 3; or cirrhosis, stage 4).

In some embodiments, the patient does not exhibit cirrhosis. In some embodiments, the patient exhibits a Maddrey discriminant function (MDF) score of ≥32 prior to treatment with the FXR agonist.

In some embodiments, the patient exhibits a model for end-stage liver disease (MELD or MELD-Na) score of ≥18 prior to treatment with the FXR agonist.

In some embodiments, the patient exhibits both an MDF score of ≥32 and a MELD or MELD-Na score of ≥18 prior to treatment with the FXR agonist.

In some embodiments, the patient exhibits an MDF score of 32 to 60.

In some embodiments, the patient exhibits a MELD score of 18 to 25 or 30 or MELD-Na score of 18 to 25 or 30.

In some embodiments, the patient exhibits a MELD score of 21 to 30, or MELD-Na score of 21 to 30.

In some embodiments, the patient exhibits an MDF score of 20 to 31 optionally in combination with a MELD score of 18 to 25 or 30 or MELD-Na score of 18 to 25 or 30.

In some embodiments, the patient exhibits an MDF score of ≥32 optionally in combination with a MELD score of 10 to 20 (or MELD-Na<21); or a MELD score of 10 to 17 (or MELD-Na<18).

In some embodiments, the patient exhibits an MDF score of ≥32 in combination with a MELD score of 21-30 (or MELD-Na 21-30).

In some embodiments, the patient exhibits an MDF score of 32, 33, 34, 35, 36, 37, 38, 39, 40, 41, 42, 43, 44, 45, 46, 47, 48, 49, 50, 51, 52, 53, 54, 55, 56, 57, 58, 59, or 60.

In some embodiments, the patient exhibits a MELD or MELD-Na score of 6, 7, 8, 9, or 10. In some embodiments, the patient exhibits a MELD or MELD-Na score of 11, 12, 13, 14, 15, 16, or 17. In some embodiments, the patient exhibits a MELD or MELD-Na score of 18, 19, 20, 21, 22, 23, 24, 25, 26, 27, 28, 29, or 30.

In some embodiments, the patient exhibits an AST of ≥50 U/L prior to treatment.

In some embodiments, the patient exhibits an AST/ALT ratio of ≥1.5 prior to treatment.

In some embodiments, the patient exhibits one or more of an AST of ≥50 U/L; an AST/ALT ratio of ≥1.5; a Maddrey discriminant function (MDF) score of ≥32; and/or a model for end-stage liver disease (MELD or MELD-Na) score of ≥18, 20 or 21, for example from 21-30.

In some embodiments, the method comprises administering to the patient in need thereof the FXR agonist in the amount of about 1 mg to about 500 mg. In some embodiments, the method comprises administering to the patient in need thereof the FXR agonist in the amount of about 1 mg to about 300 mg. In some embodiments, the method comprises administering to the patient in need thereof the FXR agonist in the amount of about 1 mg to about 200 mg. In some embodiments, the method comprises administering to the patient in need thereof the FXR agonist in the amount of about 1 mg to about 150 mg. In some embodiments, the method comprises administering to the patient in need thereof the FXR agonist in the amount of about 1 mg to about 100 mg. In some embodiments, the method comprises administering to the patient in need thereof the FXR agonist in the amount of about 1 mg to about 50 mg. In some embodiments, the method comprises administering to the patient in need thereof the FXR agonist in the amount of about 1 mg to about 25 mg. In some embodiments, the method comprises administering to the patient in need thereof the FXR agonist in the amount of about 1 mg to about 10 mg. In some embodiments, the method comprises administering to the patient in need thereof the FXR agonist in the amount of about 2.5 mg to about 500 mg. In some embodiments, the method comprises administering to the patient in need thereof the FXR agonist in the amount of about 2.5 mg to about 300 mg. In some embodiments, the method comprises administering to the patient in need thereof the FXR agonist in the amount of about 2.5 mg to about 200 mg. In some embodiments, the method comprises administering to the patient in need thereof the FXR agonist in the amount of about 2.5 mg to about 150 mg. In some embodiments, the method comprises administering to the patient in need thereof the FXR agonist in the amount of about 2.5 mg to about 100 mg. In some embodiments, the method comprises administering to the patient in need thereof the FXR agonist in the amount of about 2.5 mg to about 50 mg. In some embodiments, the method comprises administering to the patient in need thereof the FXR agonist in the amount of about 2.5 to about 25 mg. In some embodiments, the method comprises administering to the patient in need thereof the FXR agonist in the amount of about 2.5 to about 10 mg.

In some embodiments, the method comprises administering to the patient in need thereof the FXR agonist in the amount of about 2.5 mg, about 5 mg, about 10 mg, about 15 mg, about 20 mg, about 25 mg, about 50 mg, about 60 mg, about 75 mg, about 85 mg, about 100 mg, about 120 mg or about 150 mg. In some embodiments, the method comprises administering Compound 1 in the amount of about 2.5 mg, about 5 mg, about 10 mg, about 25 mg, about 50 mg, about 100 mg or about 120 mg. In some embodiments, the method comprises administering Compound 1 in the amount of about 2.5 mg, about 5 mg, about 10 mg, about 25 mg, or about 50 mg. In some embodiments, the method comprises administering to the patient in need thereof the FXR agonist in the amount of about 5 mg to about 100 mg or 5 mg to about 120 mg. In some embodiments, the method comprises administering to the patient in need thereof the FXR agonist in the amount of about 5 mg to about 50 mg. In some embodiments, the method comprises administering to the patient in need thereof the FXR agonist in the amount of about 5 to about 25 mg. In some embodiments, the method comprises administering to the patient in need thereof the FXR agonist in the amount of about 5 to about 10 mg. In some embodiments, the method comprises administering to the patient in need thereof the FXR agonist in the amount of about 5 mg. In some embodiments, the method comprises administering to the patient in need thereof the FXR agonist in the amount of about 10 mg. In some embodiments, the method comprises administering to the patient in need thereof the FXR agonist in the amount of about 25 mg. In some embodiments, the method comprises administering to the patient in need thereof the FXR agonist in the amount of about 50 mg. In some embodiments, the method comprises administering to the patient in need thereof the FXR agonist in the amount of about 100 mg.

In some embodiments, the method provides improved 90-day transplant-free survival of the patient versus an otherwise similar patient who was not administered Compound 1 or a pharmaceutically acceptable salt or amino acid conjugate thereof

In some embodiments, the method provides a reduction in Lille score at 7 days over standard of care (SOC). In some embodiments, the method reduces Lille score by 0.1, 0.2, 0.3, 0.4, 0.5, 0.6, 0.7, 0.8, or 0.9 or greater. In some embodiments, the method reduces Lille score at 7 days by 0.1, 0.2, 0.3, or 0.4. In some embodiments, the method provides a Lille score of 0.4, 0.3, 0.2, 0.1, or less than 0.1.

In some embodiments, the method provides a clinical outcome of 7-day Lille score of <0.45. In some embodiments, the method provides a clinical outcome of 7-day Lille score of <0.45 and 28-day MELD or MELD-Na score of a decrease to <21, or to <20, or to <18, or a decrease of at least 10-25%, or of at least 10-30%, or of at least 10-35%, or of at least 10-40%, (or by least 2 or 3) in combination with one, two, three, four, five, or more endpoints described below:

| | |
|---|---|
| Markers of Efficacy or | Lille score at Day 7 or 28-day (short-term) mortality; |
| Clinical Outcomes | or Lille score at Day 28 and/or Day 90; |
| | MELD or MELD-Na scores at Day 28 and/or Day 90. |
| Markers of Efficacy | MELD-Na score at Day 28 and Day 90 |
| Clinical outcomes | Reduction in occurrence of 28 day (short term) 56-day, and |
| | 84-day or 90-day (intermediate-term) mortality or liver |
| | transplantation |
| Infectious complications | Occurrence of infectious complications by SOC/PT |
| | Reduction in occurrence of infectious complications |
| | including but not limited to sepsis, and SBP during the |
| | initial hospitalization |
| Safety and Tolerability | Manageable or preferably low rate of TEAEs, SAEs, and/or |
| | acceptable or preferably healthy results of ECGs, vital signs |
| | (blood pressure, heart rate, body temperature, respiratory |
| | rate), and physical examinations, MELD-Na score |
| Clinical outcomes | Reduction in occurrence of any of the following, hospital |
| | re-admission for alcohol-associated hepatitis |
| | (hospitalization as defined by a stay of ≥24 hours) within |
| | 30 days, 60 days or 90 days |
| Measure of health care | Improvement in rates of hospitalization reason, length of |
| utilization | stay, ICU days, major medical procedures, and emergency |
| | room visits |
| Serum Liver biochemistry | ALP, ALT, AST, GGT, and total and direct bilirubin |
| Quality of Life | EQ-5D-5L |
| Markers of | Improvement towards normal levels of αIL-6, hs-CRP, CK- |
| inflammation/apoptosis | 18, and TNF-α |
| Lipid metabolism | Improvement towards normal levels of lipoproteins (LDL, |
| | HDL, VLDL), total cholesterol, triglycerides |
| Pharmacodynamic | Improvement towards normal levels of C4, FGF-19, and |
| parameters of Compound | conjugated and unconjugated endogenous bile acids |
| 1 on FXR activation | |
| Serum Markers of | Improvement towards normal levels of LBP, 16S rDNA, |
| bacterial translocation and | and stool sample for alpha-1-antitrypsin and |
| stool microbiome analysis | microbiome/metabolome analysis |

In some embodiments, the method provides an improvement in one or more of: liver biochemistry and hepatic synthetic function, inflammation, lipoprotein metabolism, farnesoid X receptor (FXR) activation markers, and bacterial translocation (e.g., lipopolysaccharide binding protein [LBP], 16S rDNA, alpha-1-antitrypsin).

In some embodiments, the dose of the patient is increased to a maximum of 50 mg per day. In some embodiments, the dose of the patient is increased to a maximum of 100 mg per day. In some embodiments, the dose of the patient is increased to a maximum of 125, 150, 175, 200, 225, 250, 275, or 300 per day.

In some embodiments, the method reduces the MELD or MELD-Na score of the patient by at least 2, by at least 3, by at least 4, by at least 5, or by at least 6 by day 28, or day 90, of treatment. In some embodiments, the method reduces the MELD or MELD-Na score of the patient by at least 2 by day 28, or day 90, of treatment. In some embodiments, the method reduces the MELD or MELD-Na score of the patient by at least 3 by day 28, or day 90, of treatment. In some embodiments, the method reduces the MELD or MELD-Na score of the patient by at least 4 by day 28, or day 90, of treatment. In some embodiments, the method reduces the MELD or MELD-Na score of the patient by at least 5 by day 28, or day 90, of treatment. In some embodiments, the method reduces the MELD or MELD-Na score of the patient by at least 6 by day 28, or day 90, of treatment. In some embodiments, the method reduces the MELD or MELD-Na score of the patient by 2 by day 28, or day 90, of treatment. In some embodiments, the method reduces the MELD or MELD-Na score of the patient by 3 by day 28, or day 90, of treatment. In some embodiments, the method reduces the MELD or MELD-Na score of the patient by 4 by day 28, or day 90, of treatment. In some embodiments, the method reduces the MELD or MELD-Na score of the patient by 5 by day 28, or day 90, of treatment. In some embodiments, the method reduces the MELD or MELD-Na score of the patient by 6 by day 28, or day 90, of treatment.

In some embodiments, the method reduces the MELD or MELD-Na score by 10-25%. In some embodiments, the method reduces the MELD or MELD-Na score by about 10%, about 15%, about 20%, or about 25%. In some embodiments, the method reduces the MELD or MELD-Na score by about 30%, 35%, 40%, or 50%. In some embodiments, the method reduces the MELD or MELD-Na score by at least 10-25%. In some embodiments, the method reduces the MELD or MELD-Na score by at least 10%, at least 15%, at least 20%, or at least 25%, or at least 30%, or at least 35%, or at least 40%.

In some embodiments, the method reduces the MDF score of the patient by at least 2, by at least 3, by at least 4, by at least 5, or by at least 6 by day 28, or day 90, of treatment. In some embodiments, the method reduces the MDF score of the patient by 5, 6, 7, 8, 9, or 10. In some embodiments, the method reduces the MDF score of the patient by 5 or more. In some embodiments, the method reduces the MDF score by 10-25%. In some embodiments, the method reduces the MDF score by about 10%, about 15%, about 20%, or about 25%. In some embodiments, the method reduces the MDF score by about 30%, 35%, 40%, or 50%. In some embodiments, the method reduces the MDF score by at least 10-25%. In some embodiments, the method reduces the MDF score by at least 10%, at least 15%, at least 20%, or at least 25%.

In some embodiments, the method reduces the MELD or MELD-Na score of the patient by at least 2-6 and/or reduces the MDF score of the patient by at least 2-6 by day 28, or day 90, of treatment. In some embodiments, the method reduces the MELD or MELD-Na score of the patient by at least 2 and/or reduces the MDF score of the patient by at least 2 by day 28, or day 90, of treatment. In some embodiments, the method reduces the MELD or MELD-Na score of the patient by at least 3 and/or reduces the MDF score of the patient by at least 3 by day 28, or day 90, of treatment. In some embodiments, the method reduces the MELD or MELD-Na score of the patient by at least 4 and/or reduces the MDF score of the patient by at least 4 by day 28, or day 90, of treatment. In some embodiments, the method reduces the MELD or MELD-Na score of the patient by at least 5 and/or reduces the MDF score of the patient by at least 5 by day 28, or day 90, of treatment. In some embodiments, the method reduces the MELD or MELD-Na score of the patient by at least 6 and/or reduces the MDF score of the patient by at least 6 by day 28, or day 90, of treatment.

In some embodiments, the method reduces the MELD or MELD-Na score of the patient by at least 5 by day 28, or day 90, of treatment. In some embodiments, the method reduces the MDF score of the patient by at least 5 by day 28, or day 90, of treatment. In some embodiments, the method reduces the MELD or MELD-Na score of the patient by at least 5 and reduces the MDF score of the patient by at least 5 by day 28, or day 90, of treatment. In some embodiments, the method provides a reduction in MELD or MELD-Na score at 28 days or 90 days. In some embodiments, the method provides a reduction in MDF score at 28 days or at 90 days.

In some embodiments, the method further comprises monitoring the blood levels of a biomarker in the patient after administration of the FXR agonist of the present disclosure. In some embodiments, the biomarker is selected from C4, FGF-19, and endogenous bile acids.

In some embodiments, the patient has alcohol-related (or alcohol-associated) liver disease (ALD), also known as alcoholic liver disease. In some embodiments, the patient exhibits jaundice.

In some embodiments, the patient exhibits one or more of the following: (1) ongoing alcohol consumption of more than 40 g of alcohol per day in women and more than 60 g of alcohol per day in men for 6 months or more, with less than 60 days of abstinence before the onset of jaundice; (2) the presence of elevated liver enzymes (aspartate aminotransferase [AST] and/or alanine aminotransferase [ALT] concentrations≥50 IU/L but ≤400 IU/L and an AST/ALT ratio of ≥1:5); and (3) worsening jaundice, with bilirubin concentrations greater than 3 mg/dL. In some embodiments, the patient is diagnosed with sAH after a liver biopsy in addition to one, two, three, or all of the preceding signs.

In some embodiments, the patient exhibits ongoing alcohol consumption of more than 40 g of alcohol per day in women and more than 60 g of alcohol per day in men for 6 months or more, with less than 60 days of abstinence before the onset of jaundice. In some embodiments, the patient exhibits the presence of elevated liver enzymes (aspartate aminotransferase [AST] and/or alanine aminotransferase [ALT] concentrations≥50 IU/L but ≤400 IU/L and an AST/ALT ratio of ≥1:5). In some embodiments, the patient exhibits worsening jaundice, with bilirubin concentrations greater than 3 mg/dL. In some embodiments, the patient exhibits the absence of any other causes of liver disease.

In some embodiments, the patient is considered ineligible for a liver transplant per generally-accepted criteria known in the art.

In some embodiments, the patient has alcoholic use disorder (AUD). AUD can be defined based on consumption of alcohol, e.g., as consuming 3 or more alcoholic drinks per day (men) or 2 or more alcoholic drinks per day (women).

In some embodiments, the patient has been treated previously for sAH with a corticosteroid. In some embodiments, the patient did not respond to the corticosteroid. In some embodiments, the patient exhibited a Lille score of more than 0.45 after 7 days or more of treatment with the corticosteroid.

In some embodiments, the patient is currently hospitalized, i.e., hospitalized prior to beginning treatment with the FXR agonist. In some embodiments, the patient is currently hospitalized and exhibits an MDF score of at least 32 and a MELD score of 18, or 20, or 21 to 30; or MELD-Na score of 18, or 20, or 21 to 25 or 30.

In some embodiments, the patient has suffered at least one relapse of sAH, i.e., has been diagnosed and/or treated at least once before for sAH.

In some embodiments, the patient exhibits one or more of the following in addition to sAH: obesity, metabolic syndrome, hepatitis C infection, or a genetic polymorphism such as patatin-like phospholipase domain protein 3, membrane bound O-acyltransferase, and transmembrane 6 superfamily member 2 genes.

In some embodiments, the patient exhibits intestinal dysbiosis.

In some embodiments, the method improves one or measures of sAH, such as intestinal dysbiosis.

In some embodiments, the method improves 30-day survival of the patient having sAH or ACLF versus an otherwise similar patient having sAH or ACLF who was not administered the FXR agonist. In some embodiments, the method improves 60-day survival of the patient versus an otherwise similar patient who was not administered the FXR agonist. In some embodiments, the method improves 90-day survival of the patient versus an otherwise similar patient who was not administered the FXR agonist. In some embodiments, the method improves the 120-day, 180-day, or 1-year survival of the patient versus an otherwise similar patient who was not administered the FXR agonist.

In some embodiments, the method improves 30-day mortality of the patient versus an otherwise similar patient who was not administered the FXR agonist. In some embodiments, the method improves 60-day mortality of the patient versus an otherwise similar patient who was not administered the FXR agonist. In some embodiments, the method improves 90-day mortality of the patient versus an otherwise similar patient who was not administered the FXR agonist. In some embodiments, the method improves the 120-day, 180-day, or 1-year mortality of the patient versus an otherwise similar patient who was not administered the FXR agonist.

In some embodiments, the improvement in survival or mortality of the patient includes an increase in survival, or a decrease in mortality, of about 10% to about 70%. Such improvements may be measured over, for example, 30 days, 60 days, 90 days, 120 days, 180 days, 1 year, or greater than 1 year. In some embodiments, the improvement in survival or mortality of the patient includes an increase in survival, or a decrease in mortality, of about 10% to about 60%, about 10% to about 50%, about 10% to about 40%, about 10% to about 30%, or about 10% to about 20%.

In some embodiments, the method provides at least a 15% reduction in mortality at 90 days compared to standard of care (SOC). In some embodiments, the method provides a 15-20% reduction in mortality at 30 days compared to SOC. In some embodiments, the method provides a 10% reduction in mortality at 6 months. In some embodiments, the method provides a 5-10% absolute reduction in mortality at 1 year.

In some embodiments, the method provides a reduction in risk of mortality for the patient. In some embodiments, the method provides a reduction in risk of mortality and health care utilization for liver-related events in patients with sAH. In some embodiments, such reductions are compared with the current standard of care, e.g., treatment with a corticosteroid such as prednisolone. In some embodiments, the FXR agonist, such as Compound 1 or a pharmaceutically acceptable salt thereof or an amino acid conjugate thereof, is indicated for first-line therapy.

In some embodiments, the method provides a decrease in the patient's health care utilization by at least 10% for 30 days, 60 days, 90 days, 120 days, 180 days, or for 1 year, or for greater than 1 year. As used herein, the term “heath care utilization” includes one or more of hospital re-admission within 30-days after discharge, number of hospitalizations, length of stay, emergency room visits, intensive care unit (ICU) days, and infectious complications (e.g., sepsis, pneumonia, urinary tract infection [UTI], cellulitis, spontaneous bacterial peritonitis [SBP]), and major medical procedures such as liver transplant. In some embodiments, the method provides a decrease in the patient's health care utilization by at least 20% for 30 days, 60 days, 90 days, 120 days, 180 days, or for 1 year, or for greater than 1 year. In some embodiments, the method provides a decrease in the patient's health care utilization by at least 30% for 30 days, 60 days, 90 days, 120 days, 180 days, or for 1 year, or for greater than 1 year. In some embodiments, the method provides a decrease in the patient's health care utilization by about 10% to about 40% for 30 days, 60 days, 90 days, 120 days, 180 days, or for 1 year, or for greater than 1 year.

In some embodiments, the method improves 90-day survival of the patient and decreases the patient's re-hospitalizations by at least 10%, or at least 20%, or between 10% and 30%.

In some embodiments, the method provides an improvement in liver-related events (such as decompensation), or hospitalization/re-hospitalization.

In some embodiments, the method provides a reduction in hospitalization by at least 15%.

In some embodiments, the method provides a reduction in progression to transplant by at least 15%.

Compound 1, also known as TC-100, is a semi-synthetic bile acid derived from CDCA, the natural ligand for the FXR. Compound 1 is the first reported example of an 11β-OH bile acid and the only known FXR specific bile acid derivative that has no activity on the bile acid G-protein coupled receptor, TGR5. The 11β-OH imparts not only FXR agonist specificity but also makes Compound 1 highly water soluble (16-fold higher than obeticholic acid or OCA), with a high critical micellular concentration like ursodeoxycholic acid, giving Compound 1 a very low liability for detergency and thus low toxicity. The compound showed a very low hepatic residence time being promptly secreted in the bile mostly as such and, therefore, localized along the entire intestinal tract. Pellicciari, R., et al. J. Med. Chem. 2016, 59, 9201-9214.

As a targeted therapy with anti-inflammatory, anti-fibrotic, and potentially anti-apoptotic effects, FXR agonist Compound 1 could represent a significant advance over systemic corticosteroid therapy, the standard of care in the treatment of patients with sAH. Compound 1 likely also decreases intestinal permeability and could provide a considerable advance over corticosteroids with respect to bacterial translocation and infectious complications seen in patients with sAH.

The targeted mechanism of action of FXR agonists such as Compound 1 represents a novel therapeutic option over the nonspecific action of corticosteroids, the current standard of care for sAH. As described in further detail herein, in one aspect, the present invention provides methods of treating alcoholic hepatitis or severe alcoholic hepatitis or ACLF, comprising administering to a patient in need thereof an effective amount of an FXR agonist described herein.

As a targeted therapy with anti-inflammatory, anti-fibrotic, and anti-apoptotic effects, FXR agonists described herein meet a long-felt need in the art for efficacious therapies for acute liver diseases such as severe alcoholic hepatitis (sAH) and acute-on-chronic liver failure (ACLF). Dysfunction of the gut-liver axis, including increased intestinal permeability, is an important driver of alcohol-induced liver damage (Sehrawat, T. S., et al. The knowns and unknowns of treatment for alcoholic hepatitis. Lancet Gastroenterol Hepatol. 2020; 5(5)494-506). Recent murine models suggest that extrahepatic FXR deficiency is critical for the development of alcohol induced fatty liver and specifically intestinal FXR may protect against steatohepatitis by maintaining gut integrity (Huang, M., et al. Enhanced alcoholic liver disease in mice with intestine-specific farnesoid X receptor deficiency. Lab Invest. 2020; 100(9):1158-68).

### Exemplary Diseases and Patient Populations—sAH

Severe alcoholic hepatitis (sAH) is an acute liver disease distinct from other alcoholic liver diseases, many of which are chronic in nature. Furthermore, sAH has distinctive histopathologic and pathophysiologic features and may be present in a patient along with underlying, chronic liver diseases such as alcoholic liver disease (ALD) (Shah N.J., Royer A, John S. Alcoholic Hepatitis. Stat Pearls. 2021 January; 1-8; hereby incorporated by reference). During sAH, endotoxin or lipopolysaccharide (LPS), collectively referred to as pathogen-associated molecular patterns (PAMPs), cross into the portal circulation and initiate the inflammatory cascade. Increased endotoxin levels in alcoholic hepatitis are caused by alcohol-induced qualitative and quantitative changes in the gut microbiota and increases in intestinal permeability. LPS activates Kupffer cells and hepatic stellate cells via toll-like receptor 4 (TLR4) and produce reactive oxygen species (ROS), proinflammatory cytokines and chemokines that together with alcohol contribute to hepatocyte damage. Other factors contributing to hepatocyte damage include alcohol-induced activation of various immune cells (i.e., neutrophils, T cells, and other leukocytes): in addition, alcohol's direct effect on adipose tissue results in the production of damage-associated molecular patterns (DAMPs).

In the majority of AH cases liver biopsy is not required. However, liver biopsy may be needed to confirm the diagnosis of AH. Findings that confirm the diagnosis of AH on liver biopsy include hepatocyte ballooning, neutrophil infiltrate and Mallory-Denk bodies, on a background of variable degrees of steatosis and fibrosis (Singal, A. K., et al. Journal of Hepatology 2019, vol. 70, 305-313). The method of the present disclosure improves one or more histologic measures of sAH, such as macrovesicular steatosis with at least one of the following: neutrophil infiltration, hepatocyte injury (ballooning), and Mallory-Derek bodies. The method of the present disclosure improves macrovesicular steatosis. The method of the present disclosure improves neutrophil infiltration. The method of the present disclosure improves hepatocyte injury (ballooning). The method of the present disclosure improves Mallory-Denk bodies.

Excessive alcohol consumption is one of the leading causes of liver disease and the seventh leading cause of premature death worldwide. Alcohol-induced liver injury can range from steatosis to alcohol-induced steatohepatitis with fibrosis to cirrhosis and hepatocellular carcinoma. Unfortunately, many alcohol-related liver disease (ALD) patients are diagnosed at more advanced stages of disease and thus data on the prevalence of early-stage disease are limited (Singal, A. K., et al. “ACG clinical guideline: Alcoholic liver disease,” Am J Gastroenterol. 2018; 113(2):175-94; hereby incorporated by reference). The best estimated combined prevalence of non-cirrhotic and cirrhotic ALD is approximately 2% of the general population in the US. Worldwide, deaths due to alcohol-induced cirrhosis account for approximately 10% of all alcohol-attributable deaths and nearly half of deaths due to chronic liver disease.

Alcoholic hepatitis (AH) (or alcohol-associated hepatitis), also referred to as symptomatic alcoholic steatohepatitis, is a clinical syndrome of acute hepatitis due to heavy alcohol consumption with distinct histopathologic findings including steatohepatitis, neutrophilic infiltration, and fibrosis (Crabb, D. W., et al., “Standard definitions and common data elements for clinical trials in patients with alcoholic hepatitis: Recommendation from the NIAAA Alcoholic Hepatitis Consortia,” Gastroenterology 2016 April; 150(4):785-90; hereby incorporated by reference). Most often, a clinical diagnosis of AH is made in a patient with the rapid development of jaundice and liver-related complications in association with documentation of persistent heavy alcohol use within 8 weeks of the onset of symptoms and exclusion of other liver diseases (Singal, A. K., et al. Am J Gastroenterol. 2018; 113(2):175-94). In AH, abnormalities that could contribute to liver injury include production of oxidative stress as a by-product of the metabolism of ethanol and the “leakage” of endotoxin through the intestinal wall and into the portal circulation. Endotoxin binds to receptors on Kupffer cells and activates an inflammatory response. The inflammatory response in the liver leads to hepatocyte dysfunction and death. Among the most important inflammatory cytokines is tumor necrosis factor (TNF)-alfa, elevated levels of which correlate with severity of liver disease. Morgan, T. R. Gastroenterol. Hepatol. (N Y). 2007 February; 3(2): 97-99.

Direct hepatocyte injury is due to the cytotoxic effect of ethanol and its metabolites (e.g., acetaldehyde). The ethanol-induced injury incites an inflammatory response and is associated with high morbidity and mortality, especially in patients with severe disease. Ethanol metabolism also changes the redox state of hepatocytes and interferes with carbohydrate and lipid metabolism, thus contributing to hepatic steatosis. Alcohol increases the susceptibility of hepatocytes to free radical damage by activating the CYP2E1 enzyme inducing mitochondrial dysfunction, depleting antioxidant stores, and recruiting inflammatory cells. Chronic alcohol abuse, particularly when combined with malnutrition, often compounds the effect of oxidative injury by further lowering cellular resilience to oxidative stress and depleting antioxidant stores. Proteasome dysfunction also serves a role in exacerbating oxidative stress and cellular injury. Secondary damage of hepatocytes, mediated by inflammatory cells, also plays a central role in the pathophysiology of alcoholic hepatitis. Chronic alcohol exposure can lead to increased gut permeability and elevated circulating pathogenic products (e.g., lipopolysaccharide), also known as pathogen-associated molecular patterns (PAMPs). Hepatocytes damaged by ethanol release aseptic inflammatory mediators known as damage-associated molecular patterns (DAMPs). DAMPs and PAMPs bind to pattern recognition receptors and potently stimulate the innate immune response. Activation of the innate immune response by cytokine-mediated and chemokine-mediated attraction of circulating neutrophils and monocytes. This leads to dense neutrophil infiltration of the liver, which is a hallmark of alcoholic hepatitis. Adaptive immune responses, mediated by B cells, T cells, and natural killer cells, also contribute to the hepatic inflammatory storm. Alcoholic hepatitis (or alcohol-associated hepatitis) is thus a distinct disease (Sehrawat, T. S., et al. “The knowns and unknowns of treatment for alcoholic hepatitis,” Lancet Gastroenterol Hepatol. 2020; 5(5)494-506; hereby incorporated by reference). Patients with sAH are defined by MDF≥32 and/or MELD>20. The short-term mortality for patients with sAH has been reported to be as high as 46% in prior interventional studies (Akriviadis, E., et al. “Pentoxifylline improves short-term survival in severe acute alcoholic hepatitis: a double-blind, placebo-controlled trial,” Gastroenterology 2000 December; 119(6): 1637-48; hereby incorporated by reference). A more recent study reported a longer term 1-year mortality (or liver transplantation) rate of 56% to 57% in patients with sAH (Thursz, M. R., et al. “Prednisolone or pentoxifylline for alcoholic hepatitis,” N Engl J Med. 2015 Apr. 23; 372(17):1619-28; hereby incorporated by reference). In support of this, intermediate mortality from AH was found to be as high as 44% at 180 days after hospital admission in a meta-analysis evaluating published mortality rates between 1971 and 2016 (Hughes, E., et al. “Survival from alcoholic hepatitis has not improved over time,” PLoS One 2018; 13(2):e0192393; hereby incorporated by reference).

Patients with acute alcoholic hepatitis will have an elevated total bilirubin and transaminases typically at least 2-6 times the upper limit or normal. Severe alcoholic hepatitis may be marked by bilirubin levels over 10-15 mg/dL; levels of aspartate aminotransferase (AST) are usually between 100 and 200 U/L (or IU/L) and are almost always less than 400 U/L (or IU/L); alanine aminotransferase (ALT) is usually approximately 25-50% the value of AST, somewhere in the range of 50-150 U/L (or IU/L); and other typical findings include fever, leukocytosis (white blood cell>10,000/μL), ascites, and tender hepatomegaly (Morgan, T. R. Gastroenterol. Hepatol. (N Y). 2007 February; 3(2): 97-99).

Current guidelines recommend alcohol abstinence in all patients with AH, as well as ALD. In addition, most patients with AH will benefit from nutritional support including total caloric and protein supplementation. However, for patients with sAH (as defined by MDF≥32 and/or MELD score>20) the treatment options are limited to the addition of systemic corticosteroids along with supportive care and alcohol abstinence.

The clinical utility of systemic corticosteroid therapy is largely limited due to the heightened risk of infection and concern for GI bleeding. Thus, all current guidelines recommend clinicians use the Lille score after 7 days of corticosteroid therapy to identify non-responders in order to avoid the risk of unnecessary systemic steroid exposure (European Association for the Study of the Liver (EASL). EASL Clinical Practice Guidelines: Management of alcohol-related liver disease. J Hepatol. 2018; 69:154-81; and Crabb, D. W., et al. “Diagnosis and treatment of alcohol-associated liver diseases: 2019 Practice guidance from the American Association for the Study of Liver Diseases,” Hepatology. 2020; 70(1): 306-33; hereby incorporated by reference).

The Lille Model is a medical modeling tool for predicting mortality in patients with alcoholic hepatitis who are not responding to steroid therapy. The model risk stratifies patients who have been receiving steroid treatment for seven days to predict who will improve and who should be considered for alternative treatment options including early referral for transplant (Mathurin, P., et al., Early change in bilirubin levels is an important prognostic factor in severe alcoholic hepatitis treated with prednisolone. Hepatology, 2003, 38 (6): 1363-9). The model is based on: Age; Albumin; Bilirubin (initial); Bilirubin (day 7); Creatinine; and Prothrombin time (PT). Recent cohort studies demonstrate that those with a Lille score above 0.45 are likely non-responders to steroid therapy (Louvet, A., et al., The Lille model: a new tool for therapeutic strategy in patients with severe alcoholic hepatitis treated with steroids, Hepatology 2007, 45 (6): 1348-54).

The Lille model can be used to predict mortality at 6 months. Lower scores indicated more improvement (in response to corticosteroids). Typically, scores>0.45 predict a 6-month survival of 25%. Scores<0.45 predict a 6-month survival of 85%. Lille scores at day 7 can range from less than 0.1 and greater than 0.9. Lille score at day 7 for Responder is <0.45 and Lille score at day 7 for non-Responder is >0.45.

Several scores such as Maddrey's discriminant function (MDF), MELD or MELD-Na, ABIC, and GAHS are used for a 28-day mortality prognosis. MDF is defined as 4.6×(patient−control PT)+serum bilirubin, with severe disease being a score of 32 or higher. An MDF score less than 32 means mild to moderate alcoholic hepatitis. An MDF score equal to or greater than 32 means one is likely to have severe alcoholic hepatitis and is a candidate for corticosteroid therapy or pentoxifylline treatment under current standard of care. MDF less than 32 (mild to moderate AH) does not benefit from steroid therapy. There is some mortality associated with mild to moderate AH, so it is beneficial to have a therapy for this group (given that steroids are not indicated because they do not impart mortality benefit).

Model for End-Stage Liver Disease (MELD) score is a prognostic scoring system, based on laboratory parameters, used to predict 3-month mortality due to liver disease. The MELD score ranges from 6 to 40 and is based on results from several lab tests. MELD scores for mild AH ranges from 6 to 10. MELD scores for moderate AH ranges from 11 to 20. MELD for sAH ranges from 21 to 30. MELD is defined as 9.57 logₑ (S creatinine)+3.78 logₑ (serum bilirubin)+11.2 logₑ (INR)+6.43. A severe disease is MELD score of 21 or higher. Singal, A. K., et al., “Diagnosis and Treatment of Alcohol-Associated Liver Disease,” JAMA. 2021; 326(2):165-176, hereby incorporated by reference.

A new version of MELD (MELD 3.0) was recently developed, which added female gender and albumin in the score, demonstrating a slightly more accurate mortality prediction than MELD-Na in cirrhotic patients. See Kim W. R., et al. MELD 3.0: The Model for End-stage Liver Disease Updated for the Modern Era. Gastroenterology 2021 (available on line at https://doi.org/10.10531j.gastro.2021.08.050), incorporated herein by reference. Each of the embodiments described herein that list MELD or MELD-Na ranges and/or values are intended to also include the corresponding MELD 3.0 ranges and/or values.

Severe AH is generally accepted to be defined by a Maddrey discriminant function (MDF) score≥32 or a MELD score≥=21, with ranges typically seen from 21-30. The MELD-Na score is a modification of the MELD score that adjusts for serum sodium. The MELD-Na score is currently an accepted criterion for determining candidacy for liver transplants. The study described herein includes patients that have MELD-Na scores of 18-25 (inclusive) and a MDF of ≥32.

Low serum sodium is an independent predictor of mortality in patients with cirrhosis. MELD-Na score includes serum Na; this score improves slightly the predictive accuracy of the MELD score in predicting mortality (Kim, W. R., et al. N Engl J Med 2008: 359:1018-1026). The MELD-Na is a validated scoring system used to assess the severity of chronic liver disease. The MELD-Na score is useful in assessing subjects with significant decompensation and is now used by the Organ Procurement and Transplantation Network (OPTN) in the US and the Eurotransplant (ET) regions of the EU to manage the organ allocation for liver transplantation. An increasing MELD-Na score is associated with increased severity of hepatic dysfunction and increased 3-month mortality risk. The MELD-Na score is derived from the subject's serum total bilirubin, serum creatinine, and INR (International normalized ratio), as appropriate, to predict survival and is calculated according to the following formula MELD-Na Score=10×[(0.957×ln(Creatinine))+(0.378×ln(Bilirubin))+(1.12×ln(INR))]+6.43; MELD-Na=MELD−Na−[0.025×MELD×(140−Na)]+140.

Patients with sAH and progressive liver failure who are non-responders to corticosteroid therapy and ineligible for liver transplantation are frequently referred for palliative care (Singal, A. K., et al. Am J Gastroenterol. 2018; 113(2):175-94). Given the limited treatment options, the side effects and lack of long-term benefit of corticosteroid therapy, there remains a significant unmet medical need for patients with sAH.

In some embodiments, the patient is of at least 18 years of age. In some embodiments, the patient is of at least 30 years of age. In some embodiments, the patient is of at least 35 years of age. In some embodiments, the patient is of at least 40 years of age. In some embodiments, the patient is of at least 60 years of age. In some embodiments, the patient is 18 to 65 years of age. In some embodiments, the patient is of at least 42, 45, 48, 50, 52, 55, 58, 62, 65, 68, 70, 72, 75, 78, or 80 years of age. In some embodiments, the patient is 40-60 years of age. In some embodiments, the patient is 60-85 years of age. In some embodiments, the patient is 40-50 years of age. In some embodiments, the patient is 70-80 years of age.

In some embodiments, the patient is male. In some embodiments, the patient is female.

In some embodiments of the present disclosure, the patient is stratified according to disease severity. Short-term mortality can be predicted using the Maddrey discriminant function (MDF), Model for End-Stage Liver Disease (MELD or MELD-Na) score, Age-Bilirubin-International Normalized Ratio-Creatinine score, and Glasgow Alcoholic Hepatitis Scores. Patients with severe AH (sAH) defined by a Maddrey discriminant function of 32 or greater have a 1-month morality rate as high as 20%-50%, so in prior clinical studies, the 30-day survival was the endpoint for most trials. The maximum MDF score is typically less than 60, as patients with MDF score of more than 60 likely need liver transplant. In some embodiments, a MELD or MELD-Na score of ≥20 or ≥21 is used as a criterion for selecting a patient. In some embodiments, a MELD or MELD-Na score of ≥18 is used as a criterion for selecting a patient. For example, a MELD score of 21 or greater predicts a 90-day mortality of 20%. The maximum MELD score is typically less than 30, as patients with MELD score of more than 30 likely need liver transplant. Failure of improvement in serum bilirubin (the Lille score) predicts patients with severe AH who are unlikely to benefit from continued corticosteroid therapy.

In some embodiments, the patient has mild alcoholic hepatitis. In some embodiments, the patient has moderate alcoholic hepatitis. In some embodiments, the patient has a Maddrey's discriminant function score of about 32, and/or a MELD or MELD-Na score of 18, 19, 20, 21, 22, 23, 24, or 25.

In some embodiments, the patient has a Maddrey's discriminant function score of ≥32, and/or a MELD or MELD-Na score of 21-30.

In some embodiments, the patient has underlying alcohol-related liver disease (ALD) associated with sAH.

In some embodiments, the patient exhibits jaundice.

In some embodiments, the patient exhibits one or more of the following: (1) ongoing alcohol consumption of more than 40 g of alcohol per day in women and more than 60 g of alcohol per day in men for 6 months or more, with less than 60 days of abstinence before the onset of jaundice; (2) the presence of elevated liver enzymes (aspartate aminotransferase [AST] and alanine aminotransferase [ALT] concentrations≥50 IU/L but ≤400 IU/L and an AST/ALT ratio of ≥1:5); and (3) worsening jaundice, with bilirubin concentrations greater than 3 mg/dL. In some embodiments, the patient is diagnosed with sAH after a liver biopsy in addition to one, two, three, or all of the preceding signs of the disease.

In some embodiments, the patient is considered ineligible for a liver transplant per generally accepted criteria known in the art.

In some embodiments, the patient has alcoholic use disorder (AUD), defined as consuming 3 or more alcoholic drinks per day (men) or 2 or more alcoholic drinks per day (women).

In some embodiments, the patient has been treated previously for sAH with a corticosteroid. In some embodiments, the patient did not respond to the corticosteroid. In some embodiments, the patient exhibited a Lille score of more than 0.45 after 7 days or more of treatment with the corticosteroid.

In some embodiments, the patient has an MDF score of 32 or higher, and/or a MELD or MELD-Na score of 18, or 20, or 21 or higher, for example 21-30.

In some embodiments, the patient has asymptomatic sAH, or “walking AH.” In some embodiments, the patient has asymptomatic sAH that includes cirrhosis.

In some embodiments, in addition to sAH, the patient is diagnosed using Child-Turcotte-Pugh criteria. In some embodiments, the patient has a Child-Turcotte-Pugh score of 7 or greater, e.g., 7-15, 8-15, 9-15, 10-15, or 8-12.

In some embodiments, the patient exhibits alcohol-associated cirrhosis.

In some embodiments, the patient exhibits alcohol-associated liver fibrosis.

In some embodiments, the patient exhibits alcohol-associated steatosis.

In some embodiments, the patient exhibits alcohol-associated steatohepatitis.

In some embodiments, the patient exhibits alcoholic hepatitis.

In some embodiments, the patient has ALD in addition to sAH, wherein the ALD is selected from early ALD (stage 0-2) or advanced ALD (bridging fibrosis, stage 3; or cirrhosis, stage 4).

In some embodiments, the patient does not exhibit cirrhosis.

In some embodiments, the patient is currently hospitalized, i.e., hospitalized prior to beginning treatment with the FXR agonist. In some embodiments, the patient is currently hospitalized and exhibits an MDF score of at least 32 and a MELD score of 18, or 20, or 21 to 25 or 30, or 21-30, or MELD-Na score of 18, 20 or 21 to 25 or 30.

In some embodiments, the patient has suffered at least one relapse of sAH.

In some embodiments, the patient exhibits one or more of the following in addition to sAH: obesity, metabolic syndrome, hepatitis C infection, or a genetic polymorphism such as patatin-like phospholipase domain protein 3, membrane bound O-acyltransferase, and transmembrane 6 superfamily member 2 genes.

In some embodiments, the patient exhibits intestinal dysbiosis.

In some embodiments, the method improves one or measures of sAH, such as intestinal dysbiosis.

In some embodiments, the present disclosure provides a method of determining candidacy of a patient for therapeutic treatment for sAH, comprising determining the patient's MELD-Na score, wherein the patient's MELD-Na score is ≥18. In some embodiments, the method further comprises determining the patient's MDF score, for example wherein the patient's MDF score is from 32 to 60 inclusive.

In some embodiments, the present disclosure provides a method of determining candidacy of a patient for therapeutic treatment for sAH, comprising determining the patient's MELD score, wherein the patient's MELD score is ≥21, for example from 21 to 30. In some embodiments, the method further comprises determining the patient's MDF score, for example wherein the patient's MDF score is ≥32, for example from 32 to 60 inclusive.

In some embodiments, the present disclosure provides a method for treating sAH comprising selecting a patient having a MELD-Na score≥18; and administering a therapeutic treatment for sAH, for example wherein the patient also has a MDF score from 32 to 60 inclusive. In some embodiments the therapeutic treatment comprises administering a pharmacologic agent to the patient, and/or a liver transplant.

In some embodiments, the present disclosure provides a method for treating sAH comprising selecting a patient having a MELD score≥21, for example from 21 to 30; and administering a therapeutic treatment for sAH, for example wherein the patient also has a MDF score≥32, for example from 32 to 60 inclusive. In some embodiments the therapeutic treatment comprises administering a pharmacologic agent to the patient, and/or a liver transplant.

In some embodiments, the method provides improvements in one or more measures of patient health outcomes. In the following embodiments of the invention, references to “a patient” or “the patient” may refer to an individual patient or to a group of patients. For example, a decrease in the patient's health care utilization may refer to an individual patient or to a group of patients.

In some embodiments, the method improves 30-day survival of the patient versus an otherwise similar patient who was not administered the FXR agonist. In some embodiments, the method improves 60-day survival of the patient versus an otherwise similar patient who was not administered the FXR agonist. In some embodiments, the method improves 90-day survival of the patient versus an otherwise similar patient who was not administered the FXR agonist. In some embodiments, the method improves the 120-day, 180-day, or 1-year survival of the patient versus an otherwise similar patient who was not administered the FXR agonist.

In some embodiments, the method improves 30-day mortality of the patient versus an otherwise similar patient who was not administered the FXR agonist. In some embodiments, the method improves 60-day mortality of the patient versus an otherwise similar patient who was not administered the FXR agonist. In some embodiments, the method improves 90-day mortality of the patient versus an otherwise similar patient who was not administered the FXR agonist. In some embodiments, the method improves the 120-day, 180-day, or 1-year mortality of the patient versus an otherwise similar patient who was not administered the FXR agonist.

In some embodiments, the improvement in survival or mortality of the patient includes an increase in survival, or a decrease in mortality, of about 10% to about 70%. Such improvements may be measured over, for example, 30 days, 60 days, 90 days, 120 days, 180 days, 1 year, or greater than 1 year. In some embodiments, the improvement in survival or mortality of the patient includes an increase in survival, or a decrease in mortality, of about 10% to about 60%, about 10% to about 50%, about 10% to about 40%, about 10% to about 30%, or about 10% to about 20%. In some embodiments, the improvement in survival or mortality of the patient includes an increase in survival, or a decrease in mortality, of about 20% to about 70%, about 20% to 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%. In some embodiments, the improvement in survival or mortality of the patient includes an increase in survival, or a decrease in mortality, of about 10%, 15%, 20%, 25%, 30%, 35%, 40%, 45%, 50%, 55%, 60%, 65%, 70%, 75%, 80%, 85%, 90%, or 95%.

In some embodiments, the method provides a reduction in risk of mortality for the patient. In some embodiments, the method provides a reduction in risk of mortality and health care utilization for liver-related events in patients with sAH. In some embodiments, such reductions are compared with the current standard of care, e.g., treatment with a corticosteroid such as prednisolone. In some embodiments, the FXR agonist, such as Compound 1 or a pharmaceutically acceptable salt thereof or an amino acid conjugate thereof, is indicated for first-line therapy.

In some embodiments, the method provides a decrease in the patient's health care utilization by at least 10% for 30 days, 60 days, 90 days, 120 days, 180 days, or for 1 year, or for greater than 1 year. As used herein, the term “heath care utilization” includes one or more of hospital re-admission within 30-days after discharge, number of hospitalizations, length of stay, emergency room visits, intensive care unit (ICU) days, and infectious complications (e.g., sepsis, pneumonia, urinary tract infection [UTI], cellulitis, spontaneous bacterial peritonitis [SBP]), and major medical procedures such as liver transplant. In some embodiments, the method provides a decrease in the patient's health care utilization by at least 20% for 30 days, 60 days, 90 days, 120 days, 180 days, or for 1 year, or for greater than 1 year. In some embodiments, the method provides a decrease in the patient's health care utilization by at least 30% for 30 days, 60 days, 90 days, 120 days, 180 days, or for 1 year, or for greater than 1 year. In some embodiments, the method provides a decrease in the patient's health care utilization by about 10% to about 40% for 30 days, 60 days, 90 days, 120 days, 180 days, or for 1 year, or for greater than 1 year.

In some embodiments, the method provides a decrease in the patient's health care utilization by about 10% to about 40% for 30 days, 60 days, 90 days, 120 days, 180 days, or for 1 year, or for greater than 1 year. In some embodiments, the method provides a decrease in the patient's health care utilization by about 20% to about 30% for 30 days, 60 days, 90 days, 120 days, 180 days, or for 1 year, or for greater than 1 year. In some embodiments, the method provides a decrease in the patient's health care utilization by about 10% to about 20% for 30 days, 60 days, 90 days, 120 days, 180 days, or for 1 year, or for greater than 1 year.

In some embodiments, the method decreases the patient's re-hospitalizations by at least 10%. In some embodiments, the method decreases the patient's re-hospitalizations by 10-80%.

In some embodiments, the method improves 90-day survival of the patient and decreases the patient's re-hospitalizations by at least 10%.

In some embodiments, the method further comprises administering the FXR agonist in combination with another therapeutic agent. In some embodiments, the method further comprises co-administering to the patient an effective amount of anti-inflammatory agent, an antibiotic, a probiotic, a fecal transplant, Zinc-supplement or a combination of any of those agents. In some embodiments, another therapeutic agent can be an anti-inflammatory agent, an antibiotic (e.g., Augmentin), an antioxidant, a probiotic, a fecal transplant, Zn-supplement or a combination of any of those agents. In some embodiments, the method comprises administering the FXR agonist and an anti-inflammatory agent. In some embodiments, the anti-inflammatory agent is a corticosteroid. For example, the corticosteroid may be selected from prednisolone (e.g., about 40 mg/d) or methylprednisolone (e.g., about 32 mg/d). In some embodiments, the additional therapeutic agent for co-administration is pentoxifylline. In some embodiments, the FXR agonist is administered in combination with corticosteroid or pentoxifylline. In some embodiments, the method further comprises co-administering to the patient an effective amount of pentoxifylline, Augmentin, bovine colostrum, corticosteroid (e.g., prednisolone or methylprednisolone), an ELAD agent, canakinumab, a TNF inhibitor, IL-22, N-acetylcysteine, metadoxine, IgG anti-LPS, a probiotic, a fecal transplant, Zn-supplement, or a combination of any of those agents.

In some embodiments, the further treatment comprises alcohol cessation and/or counseling.

### Exemplary Diseases and Patient Populations—ACLF

Acute-on-chronic liver failure (ACLF) is a syndrome characterized by acute decompensation of chronic liver disease associated with organ failures and high short-term mortality. Alcohol and chronic viral hepatitis are the most common underlying liver diseases. Up to 40%-50% of the cases of ACLF have no identifiable trigger; in the remaining patients, sepsis, active alcoholism and relapse of chronic viral hepatitis are the most common reported precipitating factors. An excessive systemic inflammatory response seems to play a crucial role in the development of ACLF. Using a liver-adapted sequential organ assessment failure score, it is possible to triage and prognosticate the outcome of patients with ACLF. The course of ACLF is dynamic and changes over the course of hospital admission. Most of the patients will have a clear prognosis between day 3 and 7 of hospital admission and clinical decisions such as evaluation for liver transplant or discussion over goals of care could be tailored using clinical scores. Bioartificial liver support systems, granulocyte-colony stimulating factors or stem-cell transplant are under investigation for medical care of this patient population; however, data are too premature to implement them as standard of care.

In some embodiments, the patient is of at least 18 years of age. In some embodiments, the patient is of at least 30 years of age. In some embodiments, the patient is of at least 35 years of age. In some embodiments, the patient is of at least 40 years of age. In some embodiments, the patient is of at least 60 years of age. In some embodiments, the patient is 18-65 years of age. In some embodiments, the patient is of at least 42, 45, 48, 50, 52, 55, 58, 62, 65, 68, 70, 72, 75, 78, or 80 years of age. In some embodiments, the patient is 40-65 years of age. In some embodiments, the patient is 65-85 years of age. In some embodiments, the patient is 40-50 years of age. In some embodiments, the patient is 70-80 years of age.

In some embodiments, the patient is male. In some embodiments, the patient is female.

In some embodiments, the patient has alcoholic use disorder (AUD), defined as consuming 3 or more alcoholic drinks per day (men) or 2 or more alcoholic drinks per day (women).

In some embodiments, the patient has ALD-related ACLF.

In some embodiments, the patient has ALD selected from early ALD (stage 0 2) or advanced ALD (bridging fibrosis, stage 3; or cirrhosis, stage 4).

In some embodiments, the patient has a Maddrey's discriminant function (MSF) score of 32 or higher, and/or a MELD or MELD-Na score of 18, 20, or 21 or higher, for example from 21-30.

In some embodiments, the patient has been diagnosed with ACLF using the Sequential [Sepsis-related] Organ Failure Assessment, APACHE (Acute Physiology and Chronic Health Evaluation), or acute-on-chronic liver failure score.

In some embodiments, the patient exhibits cirrhosis.

In some embodiments, the patient exhibits liver fibrosis.

In some embodiments, the patient exhibits steatosis.

In some embodiments, the patient exhibits alcoholic hepatitis.

In some embodiments, the patient does not exhibit cirrhosis.

In some embodiments, the patient exhibits intestinal dysbiosis.

In some embodiments, the patient exhibits one or more of the following in addition to ACLF: obesity, metabolic syndrome, hepatitis C infection, or a genetic polymorphism such as patatin-like phospholipase domain protein 3, membrane bound O-acyltransferase, and transmembrane 6 superfamily member 2 genes.

When ACLF is precipitated by sAH, same therapeutics as for sAH are used for treatment. There is an overlap in treatment options with other forms of ACLF including antibiotics, supplements, antioxidants and steroids. Antioxidants, such as N-acetylcysteine, are used in ACLF.

In some embodiments, the method of the present disclosure includes a co-administered treatment. In some embodiments, the method comprises administering the FXR agonist in combination with another therapeutic agent. In some embodiments, another therapeutic agent can be an anti-inflammatory agent, an antibiotic (e.g., Augmentin), an antioxidant, a probiotic, a fecal transplant, Zn-supplement or a combination of any of those agents. In some embodiments, the method comprises administering the FXR agonist and an anti-inflammatory agent. In some embodiments, the anti-inflammatory agent is a corticosteroid. For example, the corticosteroid may be selected from prednisolone (e.g., about 40 mg/d) or methylprednisolone (e.g., about 32 mg/d). In some embodiments, the additional therapeutic agent for co-administration is pentoxifylline. In some embodiments, the FXR agonist is administered in combination with corticosteroid or pentoxifylline. In some embodiments, the FXR agonist is administered in combination with N-acetylcysteine. In some embodiments, the further treatment comprises alcohol cessation and/or counseling.

### 2. Uses, Formulation and Administration

### Pharmaceutically Acceptable Compositions

According to another embodiment, the invention provides a composition comprising a disclosed compound and a pharmaceutically acceptable carrier, adjuvant, or vehicle. In certain embodiments, a composition of this invention is formulated for administration to a patient in need of such composition. In some embodiments, a composition of this invention is formulated for oral administration to a patient.

The term “subject” or “patient,” as used herein, means a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., hydroxypropyl methylcellulose), polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or amino acid conjugate thereof.

As used herein, the term “amino acid conjugate” refers to a conjugate of a compound of the invention with any suitable amino acid. Taurine (—NH(CH₂)₂SO₃H), glycine (—NHCH₂CO₂H), and sarcosine (—N(CH₃)CH₂CO₂H) are examples of amino acid conjugates. Suitable amino acid conjugates of the compounds have the added advantage of enhanced integrity in bile or intestinal fluids. Suitable amino acids are not limited to taurine, glycine, and sarcosine.

As defined herein, the term “metabolite” refers to glucuronidated and sulfated derivatives of the compounds described herein, wherein one or more glucuronic acid or sulfate moieties are linked to the compound of the invention. Glucuronic acid moieties may be linked to the compounds through glycosidic bonds with the hydroxyl groups of the compounds (e.g., 3-hydroxyl, 7-hydroxyl, 11-hydroxyl). Sulfated derivatives of the compounds may be formed through sulfation of the hydroxyl groups (e.g., 3-hydroxyl, 7-hydroxyl, 11-hydroxyl). Examples of metabolites include, but are not limited to, 3-O-glucuronide, 7-O-glucuronide, 11-O-glucuronide, 3-O-7-O-diglucuronide, 3-O-11-O-triglucuronide, 7-O-11-O-triglucuronide, and 3-O-7-O-11-O-triglucuronide, of the compounds described herein, and 3-sulfate, 7-sulfate, 11-sulfate, 3,7-bisulfate, 3,11-bisulfate, 7,11-bisulfate, and 3,7,11-trisulfate, of the compounds described herein.

As used herein, “pharmaceutically acceptable salt” refers to a conventional non toxic salt of a compound of the invention wherein the parent compound is modified by forming acid or base salts thereof. Suitable pharmaceutically acceptable salts according to the present disclosure can be readily determined and prepared by one skilled in the art and will include, for example, basic salts such as aluminum, calcium, lithium, magnesium, potassium, sodium, and zinc salts of Compound 1 or organic salts, such as quaternary ammonium salts of Compound 1.

The amount of a disclosed compound (i.e., active agent) that should be present in a composition for use a disclosed method or a disclosed pharmaceutical composition will generally be a therapeutically effective amount. A “therapeutically effective amount” or dose (or “effective amount”) refers to that amount of the active agent sufficient to result in a desired therapeutic result.

In certain embodiments, the effective amount refers to a titrated dosage administered during a titration period. In other embodiments, the effective amount refers to an adjusted or re-adjusted dosage administered after a titration period.

A “starting dose” as used herein refers to an initial dose provided to a patient to provide a clinical effect while minimizing onset or occurrence of an adverse effect. A starting dose can, in certain instances, be less than an amount typically administered to a patient. A starting dose is provided in an amount that is titrated or gradually increased over the course of a titration period or during the course of treatment with the FXR agonist described herein. A starting dose can, in certain instances, be more than an amount typically administered to a patient. A starting dose is provided in an amount that is titrated or gradually increased or reduced over the course of a titration period or during the course of treatment with the FXR agonist described herein to achieve the desired therapeutic result.

A “titration period” refers to a length of time for which a starting dose is administered to a patient. A titration period continues for a specified length of time, where the patient is often monitored for liver function and/or liver biochemistry as described herein.

An “adjusted dose” as used herein refers to a dose of the FXR agonist of the present disclosure or a composition thereof administered after the termination of a titration period. An adjusted dose can be increased or decreased compared to a starting dose but, as provided herein, patient tolerance and other factors described herein determine the dosage amount of an adjusted dose. A “re-adjusted dose” as used herein refers to any changed dosage amount or dose frequency of an adjusted dose in a patient.

The amount of Compound 1 or a pharmaceutically acceptable salt or amino acid conjugate thereof as provided above can refer to a starting dose administered during a titration period.

In certain embodiments, a starting daily dose of Compound 1 or a pharmaceutically acceptable salt or amino acid conjugate thereof administered to a patient described herein can be 2.5 mg. In certain embodiments, a starting daily dose of Compound 1 or a pharmaceutically acceptable salt or amino acid conjugate thereof administered to a patient described herein can be 5 mg. In certain embodiments, a starting daily dose of Compound 1 or a pharmaceutically acceptable salt or amino acid conjugate thereof administered to a patient described herein can be 7.5 mg. In certain embodiments, a starting daily dose of Compound 1 or a pharmaceutically acceptable salt or amino acid conjugate thereof administered to a patient described herein can be 10 mg. In certain embodiments, a starting daily dose of Compound 1 or a pharmaceutically acceptable salt or amino acid conjugate thereof administered to a patient described herein can be 12.5 mg. In certain embodiments, a starting daily dose of Compound 1 or a pharmaceutically acceptable salt or amino acid conjugate thereof administered to a patient described herein can be 15 mg. In certain embodiments, a starting daily dose of Compound 1 or a pharmaceutically acceptable salt or amino acid conjugate thereof administered to a patient described herein can be 20 mg. In certain embodiments, a starting daily dose of Compound 1 or a pharmaceutically acceptable salt or amino acid conjugate thereof administered to a patient described herein can be 25 mg. In certain embodiments, a starting daily dose of Compound 1 or a pharmaceutically acceptable salt or amino acid conjugate thereof administered to a patient described herein can be 50 mg. In certain embodiments, a starting daily dose of Compound 1 or a pharmaceutically acceptable salt or amino acid conjugate thereof administered to a patient described herein can be 100 mg. In certain embodiments, a starting daily dose of Compound 1 or a pharmaceutically acceptable salt or amino acid conjugate thereof administered to a patient described herein can be 120 mg.

In some embodiments, the adjusted dose of the patient can be increased to 5 mg, 10 mg, 25 mg, 50 mg, 100 mg or 120 mg. In some embodiments, an adjusted daily dose of the patient can be increased to 5 mg. In some embodiments, an adjusted daily dose of the patient can be increased to 10 mg. In some embodiments, an adjusted daily dose of the patient can be increased to 25 mg. In some embodiments, an adjusted daily dose of the patient can be increased to 50 mg. In some embodiments, an adjusted daily dose of the patient can be increased to 100 mg. In some embodiments, an adjusted daily dose of the patient can be increased to 120 mg. In some embodiments, the adjusted dose of the patient can be increased to 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg.

In certain embodiments, a starting daily dose of Compound 1 or a pharmaceutically acceptable salt or amino acid conjugate thereof administered to a patient described herein can be 120 mg, 100 mg, 50 mg, 25, mg, 20 mg, 15 mg, 12.5 mg, 10 mg, 7.5 mg, or 5 mg.

In some embodiments, an adjusted daily dose of the patient can be decreased to 100 mg. In some embodiments, an adjusted daily dose of the patient can be decreased to 50 mg. In some embodiments, an adjusted daily dose of the patient can be decreased to 25 mg. In some embodiments, the adjusted dose of the patient is decreased to 20 mg. In some embodiments, an adjusted daily dose of the patient can be decreased to 10 mg. In some embodiments, an adjusted daily dose of the patient can be decreased to 5 mg. In some embodiments, an adjusted daily dose of the patient can be decreased to 2.5 mg. In some embodiments, the adjusted dose of the patient is decreased to 15 mg, 12.5 mg, 10 mg, 7.5 mg, 5 mg, or 2.5 mg.

The amount of Compound 1 or a pharmaceutically acceptable salt or amino acid conjugate thereof can refer to an adjusted dose administered after a titration period as described herein. In certain embodiments, an adjusted dose of Compound 1 or a pharmaceutically acceptable salt or amino acid conjugate thereof administered to a patient described herein can be 2.5 mg. In certain embodiments, an adjusted dose of Compound 1 or a pharmaceutically acceptable salt or amino acid conjugate thereof administered to a patient described herein can be 5 mg. In certain embodiments, an adjusted dose of Compound 1 or a pharmaceutically acceptable salt or amino acid conjugate thereof administered to a patient described herein can be 7.5 mg. In another embodiment, an adjusted dose of Compound 1 or a pharmaceutically acceptable salt or amino acid conjugate thereof administered to a patient described herein can be 10 mg. In certain embodiments, an adjusted dose of Compound 1 or a pharmaceutically acceptable salt or amino acid conjugate thereof administered to a patient described herein can be 12.5 mg. In certain embodiments, an adjusted dose of Compound 1 or a pharmaceutically acceptable salt or amino acid conjugate thereof administered to a patient described herein can be 15 mg. In certain embodiments, an adjusted dose of Compound 1 or a pharmaceutically acceptable salt or amino acid conjugate thereof administered to a patient described herein can be 20 mg. In still another embodiment, an adjusted dose of Compound 1 or a pharmaceutically acceptable salt or amino acid conjugate thereof administered to a patient described herein can be 25 mg. In yet another embodiment, an adjusted dose of Compound 1 or a pharmaceutically acceptable salt or amino acid conjugate thereof administered to a patient described herein can be 50 mg. In yet another embodiment, an adjusted dose of Compound 1 or a pharmaceutically acceptable salt or amino acid conjugate thereof administered to a patient described herein can be 100 mg. In yet another embodiment, an adjusted dose of Compound 1 or a pharmaceutically acceptable salt or amino acid conjugate thereof administered to a patient described herein can be 120 mg. In yet another embodiment, an adjusted dose of Compound 1 or a pharmaceutically acceptable salt or amino acid conjugate thereof administered to a patient described herein can be 150 mg. In yet another embodiment, an adjusted dose of Compound 1 or a pharmaceutically acceptable salt or amino acid conjugate thereof administered to a patient described herein can be 200 mg. In yet another embodiment, an adjusted dose of Compound 1 or a pharmaceutically acceptable salt or amino acid conjugate thereof administered to a patient described herein can be 250 mg. In yet another embodiment, an adjusted dose of Compound 1 or a pharmaceutically acceptable salt or amino acid conjugate thereof administered to a patient described herein can be 300 mg.

The amount of Compound 1 or a pharmaceutically acceptable salt or amino acid conjugate thereof as provided above can refer to a re-adjusted dose administered after a titration period as described herein. In certain embodiments, a re-adjusted dose of Compound 1 or a pharmaceutically acceptable salt or amino acid conjugate thereof administered to a patient described herein can be 2.5 mg. In certain embodiments, a re-adjusted dose of Compound 1 or a pharmaceutically acceptable salt or amino acid conjugate thereof administered to a patient described herein can be 5 mg. In certain embodiments, a re-adjusted dose of Compound 1 or a pharmaceutically acceptable salt or amino acid conjugate thereof administered to a patient described herein can be 7.5 mg. In another embodiment, a re-adjusted dose of Compound 1 or a pharmaceutically acceptable salt or amino acid conjugate thereof administered to a patient described herein can be 10 mg. In still another embodiment, a re-adjusted dose of Compound 1 or a pharmaceutically acceptable salt or amino acid conjugate thereof administered to a patient described herein can be 25 mg. In yet another embodiment, a re-adjusted dose of Compound 1 or a pharmaceutically acceptable salt or amino acid conjugate thereof administered to a patient described herein can be 50 mg. In yet another embodiment, a re-adjusted dose of Compound 1 or a pharmaceutically acceptable salt or amino acid conjugate thereof administered to a patient described herein can be 100 mg. In yet another embodiment, a re-adjusted dose of Compound 1 or a pharmaceutically acceptable salt or amino acid conjugate thereof administered to a patient described herein can be 120 mg. In yet another embodiment, a re-adjusted dose of Compound 1 or a pharmaceutically acceptable salt or amino acid conjugate thereof administered to a patient described herein can be 150 mg. In yet another embodiment, a re-adjusted dose of Compound 1 or a pharmaceutically acceptable salt or amino acid conjugate thereof administered to a patient described herein can be 200 mg. In yet another embodiment, a re-adjusted dose of Compound 1 or a pharmaceutically acceptable salt or amino acid conjugate thereof administered to a patient described herein can be 250 mg. In yet another embodiment, a re-adjusted dose of Compound 1 or a pharmaceutically acceptable salt or amino acid conjugate thereof administered to a patient described herein can be 300 mg.

A titration period can be a period of time of about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29, days, 30 days, or 31 day. In certain embodiments, a titration period includes a time of about 1 week, 2 weeks, 3 weeks, 4 weeks, or 5 weeks. In another embodiment, the titration period includes a time of about 1 month, 2 months, 3 months, 4 months, 5 months, or 6 months. In another embodiment, the titration period can be longer than 6 months. In another embodiment, the titration period includes a time of about 3 days, 5 days, 7 days, 10 days, 14 days, 21 days or 28 days. For example, a titration period can be about 1 week. In another example a titration period can be about 2 weeks. In another example a titration period can be about 3 days. In still another example a titration period can be about 5 days. In yet another example a titration period can be about 5 days.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intracisternal, intrathecal, intrahepatic, intralesional and intracranial injection or infusion techniques. In some embodiments, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that arc commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topical and transdermal patches may also be used.

For topical applications, provided pharmaceutically acceptable compositions may be formulated in a suitable ointment containing the active component suspended or dissolved in one or more carriers. Carriers for topical administration of compounds of this invention include, but arc not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this invention are administered with food.

In other embodiments, pharmaceutically acceptable compositions of this disclosure are formulated for intravenous (IV) administration.

The amount of compound(s) of the present invention that may be combined with the carrier materials to produce a composition in a single dosage form will vary depending upon the host treated, the particular mode of administration. Compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Treatment, Duration and Dosing

As used herein, the term “treating” includes any effect, e.g., lessening, reducing, modulating, or eliminating, that results in the improvement of the condition, disease, disorder, etc. “Treating” or “treatment” of a disease state includes inhibiting the existing disease state, i.e., arresting the development of the disease state or its clinical symptoms, or relieving the disease state, i.e., causing temporary or permanent regression of the disease state or its clinical symptoms.

“Preventing” a disease state includes causing the clinical symptoms of the disease state not to develop in a subject that may be exposed to or predisposed to the disease state but does not yet experience or display symptoms of the disease state.

The term “inhibiting” or “inhibition” as used herein refers to any detectable positive effect on the progression of a disease or condition. Such a positive effect may include the delay in progression of at least one symptom or sign of the disease or condition, alleviation or reversal of the symptom(s) or sign(s) and slowing of the further worsening of the symptom(s) or sign(s).

Disclosed methods of treatment encompass administration of the FXR agonist of the present disclosure, or a composition thereof as needed to obtain the desired therapeutic effect. The compound or composition can be administered as long as necessary to maintain the desired therapeutic effect.

In some embodiments, the FXR agonist of the present disclosure is administered for a period (duration of treatment) between about one week and about 12 months. In some embodiments, the FXR agonist is administered for a period between about one week and about six months. In some embodiments, the FXR agonist is administered for a period between about one week and about three months. In some embodiments, the FXR agonist is administered for a period between about one week and about ten weeks. In some embodiments, the FXR agonist is administered for a period between about one week and about six weeks. In some embodiments, the FXR agonist is administered for a period between about one week and about four weeks. In some embodiments, the FXR agonist is administered for a period between about one week and about three weeks. In some embodiments, the FXR agonist is administered for a period between about one week and about two weeks. In some embodiments, the FXR agonist is administered for a period of about one week.

In some embodiments, the FXR agonist is administered to the patient for about 30 days. In some embodiments, the FXR agonist is administered to the patient for about 60 days. In some embodiments, the FXR agonist is administered to the patient for about 90 days. In some embodiments, the FXR agonist is administered to the patient for about 30 to about 90 days. In some embodiments, the FXR agonist is administered to the patient for about 30 to about 180 days. In some embodiments, the FXR agonist is administered to the patient for about 60 to about 120 days. In some embodiments, the FXR agonist is administered to the patient for about 90 to about 120 days. In some embodiments, the FXR agonist is administered to the patient for about 90 to about 180 days.

In some embodiments, the FXR agonist is administered to the patient from about 3 days to about 365 days. In some embodiments, the FXR agonist is administered to the patient for about 3 days, about 5 days, about 7 days, about 10 days, about 14 days, about 21 days, about 28 days, about 30 days, about 35 days, about 40 days, about 50 days, about 60 days, about 70 days, about 80 days, about 90 days, about 100 days, about 110 days, about 120 days, about 130 days, about 140 days, about 150 days, about 160 days, about 170 days or about 180 days.

In some embodiments, the FXR agonist is administered for about one month, about two months, about three months, about four months, about five months, about six months, about seven months, about eight months, about nine months, about ten months, about eleven months, about twelve months or as needed to bring about the desired therapeutic effect.

In some embodiments, the FXR agonist such as Compound 1 or a pharmaceutically acceptable salt or amino acid conjugate thereof is administered daily at a dose of about 1 mg to about 300 mg. In some embodiments, the FXR agonist such as Compound 1 or a pharmaceutically acceptable salt or amino acid conjugate thereof is administered daily at a dose of about 1.0 mg, about 1.5 mg, about 2.0 mg, about 2.5 mg, about 5 mg, about 10 mg, about 15 mg, about 20 mg, about 25 mg, about 30 mg, about 40 mg, about 50 mg, about 60 mg, about 75 mg, about 85 mg, about 100 mg, about 120 mg, about 125 mg, about 140 mg, about 150 mg, about 165 mg, about 175 mg, about 200 mg, about 225 mg, about 250 mg, or about 300 mg. In some embodiments, Compound 1 is administered daily at a dose of about 2.5 mg, about 5 mg, about 10 mg, about 25 mg, about 30 mg, about 35 mg, about 50 mg, about 75 mg, or about 100 mg.

In some embodiments, the FXR agonist such as Compound 1 or a pharmaceutically acceptable salt or amino acid conjugate thereof is administered in single or multiple doses sufficient to achieve a total daily dose of about 1 mg to about 150 mg.

In some embodiments, the FXR agonist such as Compound 1 or a pharmaceutically acceptable salt thereof or amino acid conjugate thereof is administered three times a day, twice a day, once a day, once every two days, once every three days, once every four days, once every five days, twice per week, once a week, or once every two weeks.

In some embodiments, the FXR agonist such as Compound 1 or a pharmaceutically acceptable salt thereof or amino acid conjugate thereof is administered at a dose of about 1 am to about 500 mg. In some embodiments, the FXR agonist such as Compound 1 or a pharmaceutically acceptable salt thereof or amino acid conjugate thereof is administered at a dose of about 2.5 mg to about 300 mg. In some embodiments, the FXR agonist such as Compound 1 or a pharmaceutically acceptable salt thereof or amino acid conjugate thereof is administered at a dose of about 2.5 mg to about 120 mg, or about 2.5 mg to about 100 mg. In some embodiments, the FXR agonist of the present disclosure is administered at a dose of about 0.1 mg to about 25 mg. In some embodiments, the FXR agonist of the present disclosure is administered at a dose of about 2.5 mg to about 50 mg. In some embodiments, the FXR agonist of the present disclosure is administered at a dose of about 2.5 mg to about 10 mg. In some embodiments, the FXR agonist of the present disclosure is administered at a dose of about 2.5 mg to about 5 mg. In some embodiments, the FXR agonist of the present disclosure is administered at a dose of about 0.5 mg to about mg. In some embodiments, the FXR agonist of the present disclosure is administered at a dose of about 0.5 mg to about 2.5 mg.

In some embodiments, the FXR agonist such as Compound 1 or a pharmaceutically acceptable salt thereof or amino acid conjugate thereof is administered at a dose of about 2.5 mg to about 120 mg, 2.5 mg to about 100 mg, about 5.0 mg to about 100 mg, about 10 mg to about 100 mg, or about 50 mg to about 100 mg. In some embodiments, the FXR agonist of the present disclosure is administered at a dose of about 2.5 mg to about 50 mg, about 10 mg to about 50 mg, or about 25 mg to about 50 mg. In some embodiments, the FXR agonist such as Compound 1 or a pharmaceutically acceptable salt thereof or amino acid conjugate thereof is administered at a dose of about 2.5 mg to about 120 mg, about 2.5 mg to about 100 mg, about 10 mg to about 100 mg, about 25 mg to about 100 mg, or about 50 mg to about 100 mg.

In some embodiments, the FXR agonist such as Compound 1 or a pharmaceutically acceptable salt or amino acid conjugate thereof is administered in single or multiple doses sufficient to achieve a total daily dose of about 2.5 mg, about 5 mg, about 10 mg, about 15 mg, about 20 mg, about 25 mg, about 50 mg, about 70 mg, about 80 mg, about 90 mg, about 100 mg, about 110 mg, about 120 mg, about 130 mg, or about 150 mg. In some embodiments, the FXR agonist of the present disclosure is administered in single or multiple doses sufficient to achieve a total daily dose about 2.5 mg, about 5 mg, about 10 mg, about 15 mg, about 20 mg, about 25 mg, about 50 mg, about 100 mg, about 120 mg, about 150 mg, about 200 mg, about 250 mg, or about 300 mg.

In some embodiments, the FXR agonist of the present disclosure is administered to the patient in a fasted state. In some embodiments, the FXR agonist is administered to the patient in a fed state.

The contents of each document cited in the specification are herein incorporated by reference in their entireties.

### EXEMPLIFICATION

### Example 1: Treatment Effects of Three Test Articles on the Development of Alcoholic Steatohepatitis in C57BL/6J Female Mice

### Objective

The objective of this study was to assess efficacy of the test articles obeticholic acid (OCA), Compound 1 and Compound 2 on liver and intestinal pathology in a mouse model of alcoholic steatohepatitis.

Compound 2 is the 3-deoxy, 11β-OH bile acid and FXR-specific bile acid derivative that has no activity on the bile acid G-protein coupled receptor, TGR5 as described in U.S. Pat. No. 10,815,267.

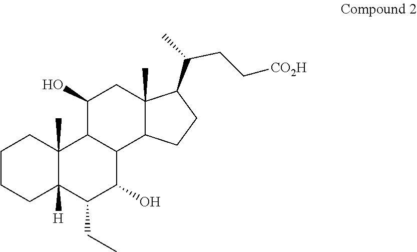 [SMILES: CC[C@H]1[C@@H](O)C2C3CC[C@H]([C@H](C)CCC(=O)O)[C@@]3(C)C[C@H](O)C2[C@@]2(C)CCCC[C@@H]12]

### Formulations and Administration of Test Articles

Vehicles for the diets used in this study are described in the table below.

TABLE 1
Formulations for Study

| | | Vehicle for | Alcohol Diet | Alcohol Diet | Alcohol Diet |
|---|---|---|---|---|---|
| | Vehicle for Gr2-7 | Alcohol Diet | Component | Component | Component |
| Identification: | 10.5% | RO water | 95% Ethanol | dry mix | Maltose |
| | Carboxymethyl | | | (F1259SP- | dextrin |
| | cellulose (CMC), | | | control diet) | |
| | medium viscosity | | | | |
| | in deionized water | | | | |
| Storage | Temperature set to | Temperature | Temperature | Temperature | Temperature |
| Conditions: | maintain 4° C. | set to maintain | set to maintain | set to maintain | set to maintain |
| | | 21° C. | 21° C. | −20° C. | 21° C. |
| Provided by: | Test Facility | Test Facility | Test Facility | Test Facility | Test Facility |

### Preparation of Formulations

Dose formulations was divided into aliquots where required to allow them to be dispensed on each dosing occasion.

TABLE 2
Preparation Details

| Dose | Administration | Frequency of | |
|---|---|---|---|
| Formulation | Dose Form | Preparation | Storage Conditions |
| Vehicle (CMC) | Solution | Monthly | Temperature set to |
| Medium Viscosity | | | maintain 2-8° C. |
| Compound 1 and | Suspension | Weekly | Temperature set to |
| OCA | | | maintain 2-8° C. |
| Compound 2 | Suspension | Every 3 days | Controlled room |
| | | | temperature |
| Alcohol Diet | Suspension | Every Other | Temperature set to |
| | | day | maintain 2-8° C. |

### Preparation of Vehicle

The vehicle, 0.5% CMC (medium viscosity) in deionized water was prepared prior to Day 1 and stored in a refrigerator set to maintain 4° C. and used for dosing appropriate groups. The vehicle was stirred at room temperature for at least 30 mins prior to dosing.

### Test Articles Preparation

Test articles for Groups 3-5 was prepared weekly, stored at 2-8° C. until ready for use and stirred at room temperature for 30 mins prior to dosing. Dose formulations was sonicated as necessary and stirred during dosing.

Test articles for Groups 6-7 was prepared every 3 days, stored at controlled room temperature until ready for use and stirred at room temperature for 30 mins prior to dosing. Dose formulations was stirred during dosing. The dosing solutions were homogenous suspensions. Any observations regarding phase separation, color, turbidity were noted.

### Preparation of Control Diet

To make 1 L of the Control Diet, weigh 225.55 gram of dry mix (F1259SP-control diet) into a lidded container. Add 350 mL of RO grade water and shake vigorously for 30 seconds. Make sure that a homogenous suspension is obtained. Add 510 mL of RO water to bring up to 1 L. A blender may be used to obtain a homogenous suspension. Control diet is administered to all groups on Day −28. The prepared diet may be stored at 4° C. and is dispensed within 3 days. The prepared diet should not be kept longer than 48 hours at room temperature to prevent diet deterioration. Additionally, the pre-weighed dry powder can be stored at 4° C. for up to one week. Prior to dispensing, the diet is stirred at room temperature for at least 30 minutes.

### Preparation of Ethanol Diet

Please refer to the following Table to prepare 1 L of alcohol diet (F1697SP-ethanol diet). A blender may be used to obtain a homogenous suspension. The prepared diet may be stored at 4° C. and is used within 48 hours. Additionally, the pre-weighed dry powder can be stored at 4° C. up to one week. Prior to dispensing, the diet was stirred at room temperature for at least 30 minutes.

TABLE 3
Preparation of Ethanol Diet

| Ethanol % in | | | | | |
|---|---|---|---|---|---|
| the diet | | Dry mix | Maltose | RO grade | 95% v/v |
| (vol/vol) | Study Day | (g) | dextrin (g) | water (ml) | ethanol (ml) |
| 1 | Day -26 | 133 | 77.1 | 900 | 10.5 |
| 2 | Day -25 | 133 | 62.9 | 910 | 21.1 |
| 3 | Day -24 | 133 | 48.7 | 910 | 31.6 |
| 4 | Day -23 | 133 | 34.5 | 920 | 42.1 |
| 5 | Day -22 to | 133 | 20.3 | 910 | 52.6 |
| | Day 15 | | | | |

### Test System

Species: Mouse
Strain: C57BL/6J (JAX #000664)
Condition: Purpose-bred, naïve
Source: Jackson Laboratories
Number of Females: 138 (119 on study+19 spares)
Target Age at the 10 weeks

### Initiation of Study:

### Animal Identification

Method: A subcutaneously implanted electronic identification chip or other approved identification method such as indelible ink where required.

### Selection, Assignment, Replacement, and Disposition of Animals

Upon arrival, all animals were fed 5CR4 chow diet ad libitum. All animals were acclimated to the vivarium for at least 4 days following arrival at the Testing Facility. Animals were weighed on Day −5 and then assigned to treatment groups based on body weight. Replacement: before the initiation of dosing, any assigned animals considered unsuitable for use in the study were replaced by alternate animals. Spare animals were transferred to study Group 1 after Study Day 1. General in-life assessments to include alternate animals until released from study. Disposition: the disposition of all animals was documented in the study records.

### Husbandry

Animals were group housed up to 3 per group per cage in polycarbonate cages including appropriate bedding. Housing set-up was as specified in the USDA Animal Welfare Act (9 CFR, Parts 1, 2 and 3) and as described in the Guide for the Care and Use of Laboratory Animals.

Diet: 5CR4 chow until Day −26; Lieber-DeCarli liquid diet Days −28 to Day 15. Upon arrival at the Testing Facility, all animals were placed on PMI Nutrition International Certified Rodent Chow No. 5CR4 ad libitum. During the chow diet phase (until Day −26), the diet in meal form may be provided to individual animals as warranted by clinical signs (e.g., broken/damaged incisors or other health changes).

On Day −28 and −27, All animals were provided both the Lieber DeCarli liquid control diet (50 mL) and PMI Nutrition International Certified Rodent Chow No. 5CR4.

On Day −26, all animals received 50 mL ethanol diet (1% v/v ethanol). The percentage of alcohol in the liquid diet was increased by 1% v/v every day until reaching 5% (v/v) on day −22 and is maintained at 5% v/v until the end of the study.

Starting Day 1 of cohort 2, animals that were triple housed received an additional bottle of 50 mL, (5% v/v ethanol). Group housed cages containing 3 animals/cage received a total volume of 100 mL (split between two bottles). The alcohol diet was stirred continuously until administration.

Fresh diet was prepared every other day from Day −28 to Day 14 and was served in the afternoon.

### Experimental Design

| | | | | | | | No. of females | | |
|---|---|---|---|---|---|---|---|---|---|
| | | Dose | Dose | Dose | | | Cohort 1 | | Cohort |
| Group | | Level | volume | Conc. | Dose | Dose | PK/ | | 2 |
| No. | Treatment | (mg/kg)^{a} | (mL/kg) | (mg/mL) | route | Frequency | BioA | Main | Main |
| 1^{b} | N/A | N/A | N/A | N/A | N/A | N/A | 4 | 2 | 12 |
| 2 | Vehicle | 0 | 10 | 0 | PO | QD | 0 | 5 | 5 |
| 3 | OCA | 30 | 10 | 3 | PO | QD | 4 | 10 | 5 |
| 4 | Compound 1 | 10 | 10 | 1 | PO | QD | 12 | 2 | 5 |
| 5 | Compound 1 | 30 | 10 | 3 | PO | QD | 6 | 8 | 5 |
| 6 | Compound 2 | 10 | 10 | 1 | PO | QD | 6 | 8 | 5 |
| 7 | Compound 2 | 30 | 10 | 3 | PO | QD | 4 | 10 | 5 |

N/A = Not Applicable,
No. = Number,
Conc. = Concentration,
QD = once a day
^{a}Based on the most recent body weight.
^{b}Baseline/start of treatment control (Necropsy for Main Study Cohort 2 on day −3 and Cohort 1-Day 2 and Cohort 2-Day 1; necropsy for PK/BioA Cohort 1 is 24 hours post dose on day 14).

### Administration of Test and Control Articles

Dose Route and timing: Oral Gavage (Group 2-7) Once Daily (Day 1-14). The first day of dosing was designated as Day 1. The dose formulations were stirred continuously during dosing. The doses were given using a syringe with attached gavage cannula.

Terminal plasma and tissue samples were gathered. Plasma was analyzed for TG, TC, ALT, AST, ALP, FGF15 (LCMS), TBIL, and endotoxin.

### Tissue Collection and Preservation

Representative samples of tissues were collected and preserved as described in the table below. Additional tissue samples were collected as needed to elucidate abnormal findings.

TABLE 4A
Tissue Collection and Preservation (Main Study) Instructions

| | | | Microscopic | | Tissue | |
|---|---|---|---|---|---|---|
| Tissue | Weigh | Collect | Evaluation | Splits | part | Comment^{a} |
| Liver | X | X | — | Sp 1 | the left lateral | Total liver with gall bladder is |
| | | | | | lobe (~100 | harvested, weighed. |
| | | | | | mg +/− 10 mg) | The left lateral lobe is snap-frozen in liquid |
| | | | | | | nitrogen. The samples is stored at −80° C. |
| | | | | | | and shipped for Nanostring analysis. |
| | | | | Sp 2 | Half of | Half of right lobe (distal part) is collected |
| | | | | | right lobe | in 10% NBF. Samples fixed in NBF for |
| | | | | | | 24-48 hrs, then changed over to 70% EtOH |
| | | | | | | before transferring to histopathology lab |
| | | | | | | at Testing Facility. |
| | | | | Sp 3 | the left lateral | Record exact weight, the left lateral lobe is |
| | | | | | lobe (~50 | snap-frozen in liquid nitrogen. The samples |
| | | | | | mg +/− 10 mg) | is stored at −80° C. at the Testing Facility |
| | | | | | | for liver TG, TC assay. |
| | | | | Sp 4 | Remaining | Remove the gall bladder, record exact |
| | | | | | liver | weight, the remaining liver is snap-frozen |
| | | | | | | and stored at the Testing Facility. |
| Ileum | X | X | — | Sp 1 | One piece | Ileum is flushed with sterile saline and |
| | | | | | (~100 mg +/− | weighed and split into 2 pieces. |
| | | | | | 10 mg) | One piece is snap-frozen in liquid nitrogen. |
| | | | | | | The samples is stored at −80° C. and |
| | | | | | | shipped for Nanostring analysis. |
| | | | | Sp 2 | Second piece | The other piece is collected in 10% NBF. |
| | | | | | (remains tissue) | Samples is fixed in NBF for 24-48 hrs, |
| | | | | | | then changed over to 70% EtOH before |
| | | | | | | transferring to histopathology lab |
| | | | | | | at Testing Facility. |

X = Procedure to be conducted; — = Not applicable.
^{a}All liver splits is collected prior to flushing the ileum.

TABLE 4B
Tissue Collection and Preservation (PK/BioA)

| | | | Microscopic | |
|---|---|---|---|---|
| Tissue | Weigh | Collect | Evaluation | Comment |
| Liver | X | X | — | Liver is harvested and weighed then snap- |
| | | | | frozen in liquid nitrogen. The samples |
| | | | | is stored at −80° C. until analysis. |
| Ileum | X | X | — | Ileum is harvested, flushed, and |
| | | | | weighed then snap-frozen in liquid |
| | | | | nitrogen. The samples is stored |
| | | | | at −80° C. until analysis. |

X = Procedure to be conducted; — = Not applicable.

Frozen liver samples from main study were analyzed in duplicates for the estimation of liver lipids including triglycerides and cholesterol. Liver samples were kept on dry ice until transferred to sample management and stored in a freezer set to maintain 80° C. until analysis.

### Histology

The liver and small intestine collected from all animals in Groups 1-7 (Main Study) were fixed in 10% neutral buffered formalin for 24-48 hours and transferred to 70% EtOH, stored under ambient conditions and transferred to the histopathology lab at Testing Facility.

### Liver Tissue

Formalin fixed liver tissues were trimmed to slides and stained with H&E and picrosirius red stains. Slides were scored by Charles River board certified veterinarian pathologists.

### Small Intestine Tissue

Formalin fixed small intestine tissues were trimmed to slides and stained with H&E and IHC antibodies (PV-1, ZO-1 and Occludin). Slides were scored by board certified veterinarian pathologists.

### Results

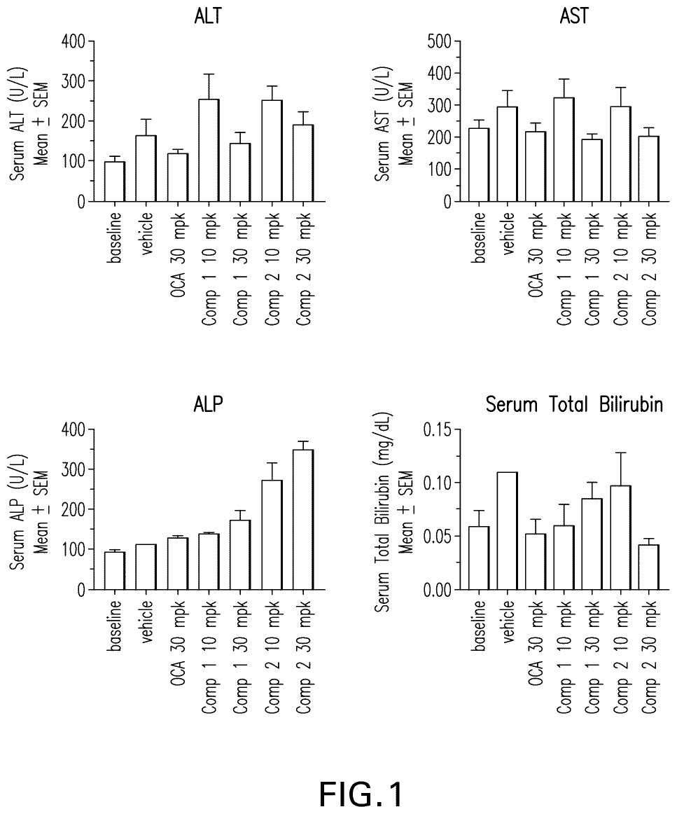

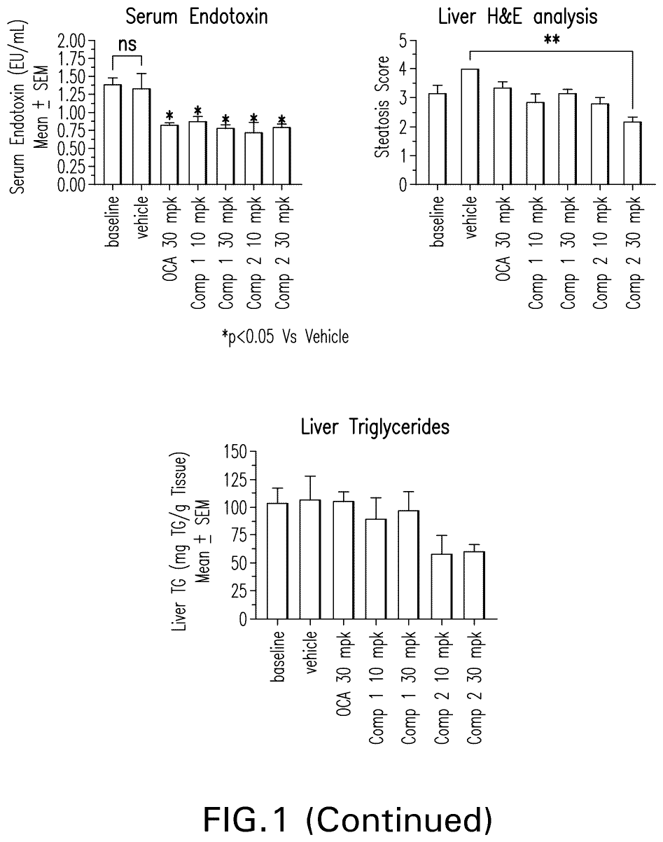

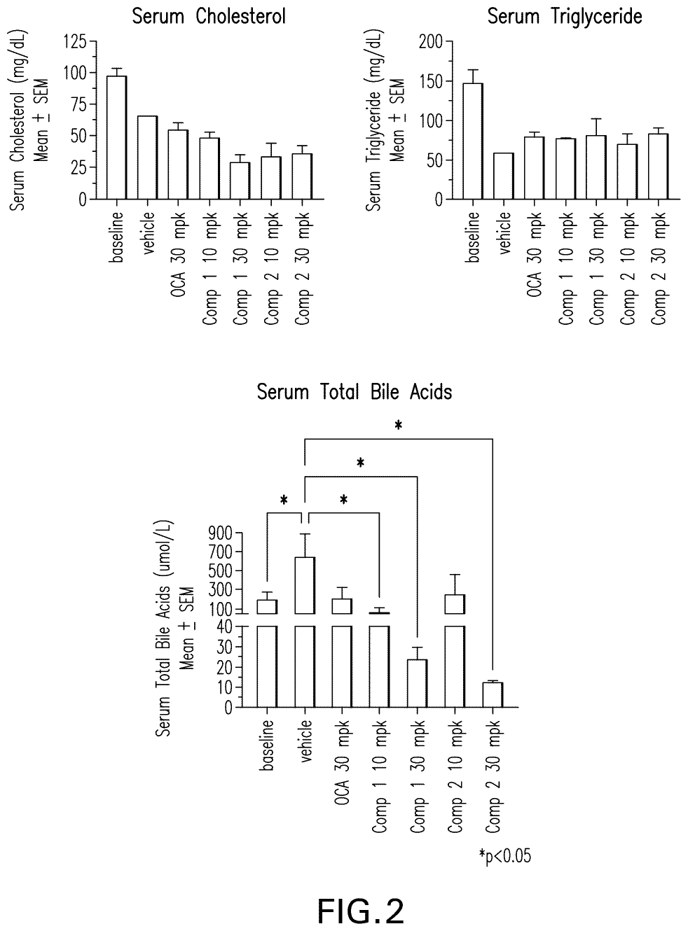

As shown in the table below, Compound 1 improved survival better than OCA. Compound 2 (Comp 2 in FIGS. 1 and 2) increased serum ALP but decreased bilirubin. The reduction in serum endotoxin is significant. For steatosis (graded 1-5 by a pathologist), the Compound 1 10 mpk group was close to statistical significance. For fibrosis as analyzed by Pico Sirius Red, the animal model did not have fibrosis at baseline or in the vehicle group. A minimal increase in fibrosis was seen in OCA 30 mpk, Compound 1 30 mpk and Compound 2 30 mpk. For exploratory endpoints for ileum, all H&E sections IHC stains for tight junctions were normal and the same between groups. See FIGS. 1 and 2 for clinical chemistry results and liver and ileum histology from this study. Without wishing to be bound by theory, it is believed that these results suggest improved gut function with all compounds, and greatly improved survival for the 10 mg/kg and 30 mg/kg Compound 1 (Comp 1 in FIGS. 1 and 2) groups that was superior to the results with the other two compounds.

TABLE 5
Results of Study

| Treatment | Dose mkd | % * Alive |
|---|---|---|
| Vehicle | 0 | 60 |
| OCA | 30 | 53 |
| Compound 1 | 10 | 79 |
| Compound 1 | 30 | 68 |
| Compound 2 | 10 | 63 |
| Compound 2 | 30 | 53 |

* Includes animals for PK

As shown in FIG. 2, Serum bile acids were markedly increased in vehicle treated ALD mice and were significantly lowered by both doses of Compound 1 (Comp 1) and the high dose of Compound 2 (Comp 2).

### Example 2: a Phase 2a, Randomized, Double-Blind, Placebo-Controlled, Multicenter, Dose-Escalation, Proof-of-Concept Study Evaluating the Safety, Tolerability, Efficacy and Pharmacokinetics of Compound 1 in Subjects with Severe Alcohol-Associated Hepatitis

Compound us a modified bile acid with high potency and selectivity for the farnesoid X receptor (FXR). Due to its FXR-mediated effects, Compound 1 is a novel therapy for alcohol-associated hepatitis. We describe below a Phase 2 proof-of-concept study evaluating the safety, tolerability, efficacy, and pharmacokinetics of Compound 1 in patients with severe alcohol-associated hepatitis (sAH).

This is a Phase 2a, randomized, double-blind, placebo-controlled, multicenter (approximately 25 sites globally), dose-escalation, proof-of-concept study evaluating the safety, tolerability, efficacy, and PK of Compound 1 in subjects, initially admitted to the hospital, with sAH.

Compound 1 is administered daily in capsule form at escalating doses starting at 5 mg. and increasing stepwise.

If there are no clinically significant safety or tolerability concerns, and after review and approval by the Sponsor's Global Safety Committee (GSC), a decision will be made to escalate to the next dose cohort and the same stepwise process will be followed (e.g., 10, 25, 50, and 100 mg (5 dose cohorts)). The dose levels for the remaining dose additional cohorts. will be defined based on the safety and tolerability data of Compound 1 from the preceding cohorts. Dose escalations will not exceed a 3-fold increase from cohort to cohort and a maximum dose level of 120 mg.

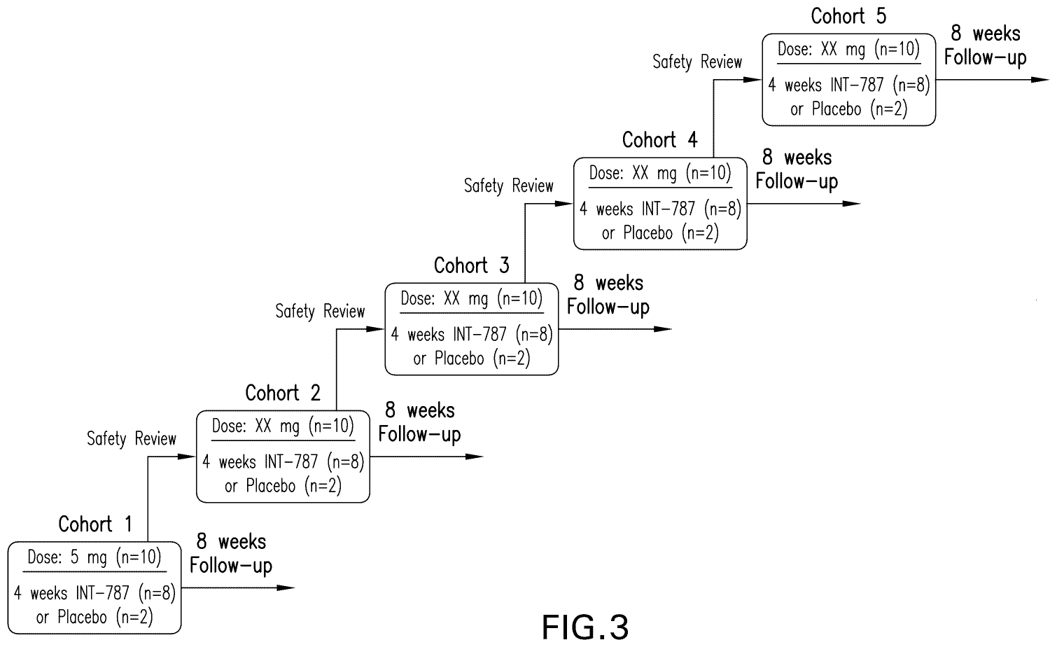

About 50 subjects aged 18 to 65 years (inclusive) with sAH (defined by a Maddrey discriminant function [MDF]≥32 and ≤60 and a model for end-stage liver disease-sodium [MELD-Na] score of 18 to 25 [inclusive]) are enrolled in the study, including about 50 subjects in the Compound 1 treatment groups. Five dose cohorts are planned with doses ranging from 5 mg to 120 mg Compound 1 (or Comp 1) (FIG. 3). Each dose cohort is comprised of 2 groups containing a total of 10 subjects, with 8 subjects randomized to study treatment (Treatment Group) and 2 subjects to placebo (Control Group). Assuming the responder rates based on the Lille model are 90% in the Compound 1 Response Cohorts and 25% in the Control Cohort, a sample size of 8 subjects per arm provides >80% power to detect treatment differences between the Response Cohorts and the Control Cohort with a 2-sided alpha of 0.05.

The primary objective of the study is to evaluate the effect of Compound 1 treatment on sAH disease progression, as assessed by the Lille score on Day 7. Secondary objectives include evaluation of the effect of Compound 1 treatment on the MELD-Na score at Day 28, mortality at 56 and 84 days (short-term and intermediate-term mortality) or liver transplantation, and any infectious complications, as well as evaluation of the PK of Compound 1 and its metabolites glyco-Compound land tauro-Compound 1 and evaluation of the safety and tolerability of Compound 1 in the proposed patient population. Safety and tolerability are assessed by recording adverse events, laboratory assessments, ECGs, vital signs, and physical examinations. Exploratory objectives/measures include (1) occurrence of hospital re-admission for alcohol-associated hepatitis (hospitalization as defined by a stay of ≥24 hours) during the study period; (2) hospitalization reason, length of stay, ICU days, major medical procedures, and emergency room visits; (3) change from Baseline in serum liver biochemistries (ALP, AST, ALT, GOT, and total and direct bilirubin); (4) measurement of health-related quality of life (EQ-5D-5L), (5) change from Baseline in IL-6, hs-CRP, CK-18, and TNF-α at Day 28; (6) change from Baseline in Lipoproteins (LDL, HDL, VLDL), total cholesterol, and triglycerides at Day 28; and (8) change from Baseline in lipid metabolism (LBP), 16S rDNA and stool sample for alpha-1-antitrypsin and microbiome/metabolome analysis.

The Control Group (placebo-treated subjects) in the study provides useful information on efficacy and safety outcomes in this patient population with severe alcohol-associated hepatitis treated with standard of care. The placebo-treated subjects within cohorts are meant to blind the study drug administration while the data across dose cohorts will be used for the overall analysis.

The dose levels for this Phase 2a clinical study are based on the overall safety and tolerability profile of Compound 1 established in the nonclinical program and the dose selection in this study is based on data from Compound 1 phase 1 dose-escalation study. There were no adverse safety signals in nonclinical safety pharmacology studies with Compound 1. Compound 1 did not alter neurologic function in an Irwin study in male rats when tested up to 180 mg/kg. Respiratory function in dogs was not affected by single doses of Compound 1 up to 300 mg/kg. Results from in vitro human ether-a-go-go-related gene (hERG) studies and in vivo studies in dogs dosed up to 300 mg/kg indicated that acute cardiovascular effects in humans are not expected. Testing of Compound 1 in an in vivo mouse bone marrow micronucleus assay and a liver comet assay showed no evidence of DNA damage up to doses of 2000 mg/kg/day for 3 days. Based on these results, Compound 1 is not considered genotoxic. In the Phase 1 study, no AEs of clinical concern were observed with doses less than 50 mg in healthy subjects who were administered single or repeat doses of Compound 1. The 5 mg dose taken once daily for 14-days was well tolerated.

The study is designed to assess increasing doses of Compound 1 in a stepwise fashion (FIG. 3). The decision to progress to the next higher dose cohort is made after a clinical review of safety and tolerability. The study design includes five Compound 1 dose cohorts. Each dose cohort is comprised of 2 groups containing a total of 10 subjects, with 8 subjects randomized to study treatment (Treatment Group) and 2 subjects to placebo (Control Group). Safety and tolerability is evaluated by examining the incidence of unexpected treatment emergent serious adverse events (SAEs), investigational product discontinuations, and the incidence of potential drug induced liver injury (DILI) at each dose. Additional considerations are given to overall treatment-emergent adverse events (TEAEs), clinical laboratory tests, ECGs, and vital signs. An increase in the Lille score to >0.85 represents potential drug ineffectiveness (rather than liver injury) and is taken into consideration as part of each dose escalation assessment. Given the acute and progressive nature of sAH, it is important to monitor for potential drug induced liver injury, and/or hepatic decompensation. MELD-Na scores is reviewed at each visit where labs are drawn.

Primary Efficacy Analyses: The primary efficacy analyses is conducted using the ITT population. The primary efficacy endpoint is the Lille score at day 7. For the analysis of Lille score, all subjects with a Day 7 Lille score will be included. The Lille score is analyzed as a categorical variable. In the primary efficacy analyses, subjects with Lille score<0.45 is counted as responders and those with Lille score>=0.45 is counted as non-responders. The percentage of responders and non-responders at day 7 is summarized by treatment group and compared using Fisher Exact test. An additional descriptive analysis of Lille score as a continuous variable is performed. The efficacy evaluation will start on the highest tolerated dose and go down to lower dose levels using the sequential approach. If the higher dose level is statistically significant on the primary efficacy endpoint, the next lower dose level is evaluated. Otherwise, the efficacy evaluation is stopped on the current dose level. The primary comparison of efficacy evaluation is on the highest tolerated dose.

Secondary and Exploratory Efficacy Analyses: The secondary and exploratory efficacy analyses is conducted using the ITT population. All continuous and categorical efficacy endpoints is summarized using descriptive statistics at Baseline and at each scheduled post-Baseline visit. For continuous endpoints, the change from Baseline will also be summarized. Analyses of continuous endpoints is carried out using a mixed-effect repeated measures model to evaluate the effect of treatment groups over time based on ITT population. The dependent variable is the change from Baseline. The model will include the terms of treatment group, visit, treatment by visit interaction as factors and Baseline value as covariate. Analyses for categorical endpoints is performed using a Fisher Exact test. Any subject who has an unknown death status at the specified time point will be excluded from the analyses of mortality. In addition, the Lille score at day 7 will also be summarized as a continuous variable by treatment group and compared using an ANOVA model with Lille score as the dependent variable including treatment group as a fixed effect.

### Objectives

### Primary Objective:

To evaluate the efficacy of Compound 1 as assessed by disease progression in severe alcohol-associated hepatitis (sAH)

### Secondary Objectives:

To evaluate the effect of Compound 1 on efficacy as assessed by the Model for End-Stage Liver Disease-Sodium (MELD-Na) score in sAH
To evaluate the effect of Compound 1 on clinical outcomes as assessed by short-term and intermediate-term mortality and/or liver transplantation in sAH
To evaluate the effect of Compound 1 on infectious complications in sAH
To evaluate the pharmacokinetics (PK) of Compound 1 and its metabolites in subjects with sAH
To evaluate the safety and tolerability of Compound 1 in subjects with sAH

Exploratory Objectives—To evaluate the effect of Compound 1 on:
Hospital re-admission due to alcohol-associated hepatitis during the study period
Measures of health care utilization
Health-related quality of life
The following measures:
   Liver biochemistry and hepatic synthetic function, renal function, hematology
   Inflammation
   Lipid metabolism
   Farnesoid X receptor (FXR) activation
   Microbiome/metabolome
   Bacterial translocation

### Diagnosis and Main Criteria for Inclusion

### Subject Inclusion Criteria

1. Males or females aged 18 to 65 years (inclusive)
2. Clinical diagnosis of sAH based on all the following:
   a.

$$History of excess alcohol ( > 60 g / day [ male ] or > 40 g / day [ female ] ) use for \geq 6 months , with < 60 days of abstinence prior to the onset of jaundice$$

b.

$$Serum total bilirubin > 3. mg / dL$$

c.

$$AST \geq 50 U / L ( or IU / L )$$

d.

$$AST / ALT ratio \geq 1.5$$

e.

$$MDF \geq 32 and \leq 60$$

f. MELD-Na score 18, or 20 or 21 to 25 (inclusive)
3. Onset of jaundice within 8 weeks from the time of admission to the hospital
4. Up to and no more than (NMT) 7 days since admission to the hospital
5. Female subjects must be postmenopausal, surgically sterile, or, if premenopausal (and not surgically sterile), be prepared to use ≥1 highly effective method of contraception during the study and for 90 days after the last dose of investigational product as follows: Surgical sterilization (bilateral tubal occlusion, etc.); Placement of an intrauterine device (IUD) or intrauterine system (e.g., intrauterine hormone-releasing system [IUS]); Combined (estrogen and progesterone containing) hormonal contraceptive associated with inhibition of ovulation: Oral; Intravaginal; Transdermal; Progesterone-only hormonal contraception associated with inhibition of ovulation: Oral; Injectable; Implantable; Sexual abstinence, if in line with the preferred and usual lifestyle of the subject (where abstinence is defined as refraining from heterosexual intercourse during the entire period of risk associated with study treatments).
6. Male subjects who are sexually active with female partners of childbearing potential must agree to use a condom with spermicide and to use one other approved method of highly effective contraception from the time of investigational product administration for at least 90 days after the dose of investigational product as listed in Inclusion Criteria 5
7. Male subjects must refrain from sperm donation from Screening through at least 90 days following the last dose of investigational product
8. Must provide written informed consent and agree to comply with the study protocol. In subjects with hepatic encephalopathy which may impair decision-making, consent is obtained per hospital procedures (e.g., by Legally Authorized Representative).

Subjects must agree to participate in an alcohol use disorder (AUD) program during the study period, as recommended by the local institution's addiction medicine specialists, including post-hospitalization.

### Subject Exclusion Criteria

1. Subjects taking systemic corticosteroids or products containing obeticholic acid (OCA) in the 30 days prior to screening, up to and including randomization.
2. Pregnancy, planned pregnancy, potential for pregnancy (i.e., unwillingness to use effective birth control during the study), or current or planned breast feeding
3. Cessation of alcohol consumption for ≥2 months before Day 1
4.

$$AST or ALT > 400 U / L$$

5.

$$MDF < 32 or > 60 at Screening$$

6.

$$MELD - Na Score < ( less than ) 18 or > 25 at Screening ( confirmed by repeat labs within 48 hours )$$

7. Other causes of liver disease including chronic hepatitis B (hepatitis B surface antigen [HBsAg] positive), chronic hepatitis C (HCV) RNA positive, acetaminophen hepatotoxicity, biliary obstruction, and autoimmune liver disease
8. Current or previous history of hepatocellular carcinoma (HCC)
9. History of liver transplantation or currently listed for liver transplant
10. Untreated sepsis (e.g., has not initiated appropriate medical treatment for infection and/or septic shock)
11. Known positivity for human immunodeficiency virus infection
12. Uncontrolled gastrointestinal (GI) bleeding or controlled GI bleeding within
7 days of Screening that was associated with shock or required transfusion of more than 3 units of blood
13. Kidney injury defined as a serum creatinine>133 μmol/L (>1.5 mg/dL) confirmed by repeat testing within 48 hours or the requirement for renal replacement therapy
14. Portal vein thrombosis
15. Acute pancreatitis or acute gallbladder disease (e.g., cholecystitis)
16. Severe associated disease (e.g., cardiac failure, acute myocardial infarction, severe cardiac arrhythmias, severe pulmonary disease, neurologic disease)
17. Malignancy within the 2 years prior to Screening, with the exception of specific cancers that have been cured by surgical resection (e.g., basal cell skin cancer). Subjects under evaluation for possible malignancy are not eligible
18. Positive Urine Drug Screen (amphetamines, barbiturates, benzodiazepines, cocaine and opiates) except tetrahydrocannabinol (THC) or in the setting of documented prescription medications (e.g., opiates, benzodiazepines, amphetamines, barbiturates, including medications prescribed as part of in-patient management. Subjects being treated for alcohol withdrawal may be exempt, verify with Medical Monitor)
19. Participated in a clinical research study and received any active investigational product being evaluated for the treatment of sAH 3 months before Day 1
20. Participation in a study of another investigational medicine or device within
30 days before Screening
21. Any other condition or clinical laboratory result that, in the opinion of the Investigator, might confound the results, or would impede compliance or hinder completion of the study
22. Received a positive SARS Coronavirus 2 (SARS-CoV-2) test within 4 weeks of Screening or a SARS-CoV-2 vaccination within 2 weeks of Screening

### Investigational Product, Dosage and Mode of Administration

The investigational product, Compound 1, capsule(s) and matching placebo capsule(s) for oral administration once daily. The investigational product, Compound 1, is supplied in multiple capsule strengths ranging from 5 mg to 120 mg. Multiple Compound 1 capsules may be taken to achieve the appropriate Compound 1 dose levels (e.g., 10 mg, 25 mg, 50 mg, and 100 mg). Matching placebo capsule(s) will also be provided for blinding purposes. This study is conducted in a double-blind (subjects and site staff) manner, randomized in a 4:1 fashion for each dose cohort.

### Reference Therapy, Dosage and Mode of Administration

All eligible subjects will receive the appropriate supportive measures, including nutritional support, per the local standard of care. Corticosteroid use will not be allowed 30 days prior to screening and up to and including randomization, as it is intended for a subject to complete the study without the use of corticosteroids, if possible (ophthalmic steroids and low-dose topical or low-dose intranasal steroids are allowable).

If systemic corticosteroids are initiated during the screening period, the subject will not be considered eligible for randomization. If systemic corticosteroids are initiated within the first 7 days post-randomization, the subject will be discontinued and replaced in that study cohort. The use of systemic corticosteroids post-randomization can be considered on a case-by-case basis if clinically warranted (e.g., Lille score>0.56 with rising total bilirubin at Day 7 or MELD-Na score increases >3 points, not due to acute kidney injury).

### Duration of Treatment

Subjects will receive investigational product for 28 days.

### Criteria for Evaluation

The endpoints supporting the primary, secondary, and exploratory objectives of the study are as follows:

Primary Endpoint

| | |
|---|---|
| Markers of Efficacy | 7-day Lille score |

Secondary Endpoints

| | |
|---|---|
| Markers of Efficacy | MELD-Na score at Day 28 |
| Clinical outcomes | 28-day (short term), 56-day, and 84-day (intermediate-term) |
| | mortality or liver transplantation |
| Infectious complications | Occurrence of infectious complications by SOC/PT |
| Compound 1 PK | Compound 1, tauro-Compound 1, glycol-Compound 1 and other |
| | metabolites as applicable |
| Safety and Tolerability | TEAEs, SAEs, laboratory assessments, ECGs, vital signs (blood |
| | pressure, heart rate, body temperature, respiratory rate), physical |
| | examinations, MELD-Na score. |

Exploratory Endpoints

| | |
|---|---|
| Clinical outcomes | Occurrence of any of the following, hospital re-admission |
| | (hospitalization as defined by a stay of ≥24 hours) during study |
| | period. |
| Measure of health care | Hospitalization reason, length of stay, ICU days, major medical |
| utilization | procedures, and emergency room visits |
| Liver biochemistry and | ALP, ALT, AST, GGT, and total and direct bilirubin |
| synthetic function | |
| Quality of Life | EQ-5D-5L |
| Markers of | αIL-6, hs-CRP, CK-18, and TNF-α |
| inflammation/apoptosis | |
| Lipoprotein | Lipoproteins (LDL, HDL, VLDL), total cholesterol, triglycerides |
| metabolism | |
| Pharmacodynamics of | C4, FGF-19, and conjugated and unconjugated endogenous bile |
| Compound 1 on FXR | acids |
| activation | |
| Serum Markers of | LBP, 16S rDNA, stool sample for alpha-1 antitrypsin and |
| bacterial translocation | microbiome/metabolome analysis |
| and stool microbiome | |
| analysis | |
| Urine Biomarkers | NGAL, KIM-1, IL-18, L-FABP |

C4 = 7α-hydroxy-4-cholesten-3-one;
CK-18 = cytokeratin 18;
hs-CRP = high sensitive c-reactive protein;
rDNA = ribosomal deoxyribonucleic acid;
ECG = electrocardiogram;
FGF-19 = fibroblast growth factor 19;
GGT = gamma glutamyl transferase;
HDL = high density lipoprotein;
ICU = intensive care unit;
IL-6 = interleukin 6;
IL-18 = interleukin 18;
KIM-1 = kidney injury molecule-1;
L-FABP = liver-type fatty acid-binding protein;
INR = international normalized ratio;
LBP = lipopolysaccharide-binding protein;
LDL = low density lipoprotein;
MELD-Na = model of end-stage liver disease-sodium;
NGAL = neutrophil gelatinase-associated lipocalin;
PK = pharmacokinetics;
SAE = severe adverse event;
SBP = spontaneous bacterial peritonitis;
TEAE = treatment-emergent adverse event;
TNF-α = tumor necrosis factor-alpha;
VLDL = very low-density lipoprotein

### Example 3: a Phase 2b/3, Randomized, Double-Blind, Placebo-Controlled, Study Evaluating the Safety, Tolerability, Efficacy and Pharmacokinetics of Compound 1 in Subjects with Severe Alcohol-Associated Hepatitis

We describe below a Phase 2b/3 study evaluating the safety, tolerability, efficacy, and pharmacokinetics of Compound 1 in patients with severe alcohol-associated hepatitis (sAH).

This is a Phase 2b/3, randomized, double-blind, placebo-controlled, multicenter (approximately 25 sites globally), dose-escalation, proof-of-concept study evaluating the safety, tolerability, efficacy, and PK of Compound 1 in subjects, initially admitted to the hospital, with sAH.

About 50 subjects aged 18 to 65 years (inclusive) with sAH (defined by a Maddrey discriminant function [MDF]≥32 and a model for end-stage liver disease [MELD] score of 21-30 [inclusive]) are enrolled in the study, including about 50 subjects in the Compound 1 treatment groups. Dose cohorts (2, 3, 4 or 5 cohorts) are planned with doses ranging from 5 mg to 120 mg Compound 1 (or Comp 1). Each dose cohort is comprised of 2 groups containing a total of 10 subjects, with 8 subjects randomized to study treatment (Treatment Group) and 2 subjects to placebo (Control Group). Assuming the responder rates based on the Lille model are 90% in the Compound 1 Response Cohorts and 25% in the Control Cohort, a sample size of 8 subjects per arm provides >80% power to detect treatment differences between the Response Cohorts and the Control Cohort with a 2-sided alpha of 0.05.

The study is generally carried out in accordance with the objectives, procedures, inclusion and exclusion criteria, efficacy analyses, criterial for evaluation, dosing amounts and regimens described above in Example 2, modified as follows: The primary objective of the study is to evaluate the effect of Compound 1 treatment on 90-day transplant-free survival of patients with sAH. Secondary objectives include evaluation of the effect of Compound 1 treatment on mortality at 28 days (short-term mortality), the MELD and Lille scores at Days 28 and 90, and evaluation of the safety and tolerability of Compound 1 in the proposed patient population at Days 28 and 90, and at 6 months. Safety and tolerability are assessed by recording adverse events, laboratory assessments, ECGs, vital signs, and physical examinations. Exploratory objectives/measures include (1) mortality at 6-months; 2) healthcare utilization including: occurrence of hospital re-admission for alcohol-associated hepatitis (hospitalization as defined by a stay of ≥24 hours) during the study period; hospitalization reason, length of stay, ICU days, major medical procedures, and emergency room visits; (3) evaluation of pharmacokinetic and pharmacodynamic biomarkers, including: change from Baseline in serum liver biochemistries (ALP, AST, ALT, GGT, and total and direct bilirubin); change from Baseline in IL-6, hs-CRP, CK-18, and TNF-α at Day 28; change from Baseline in Lipoproteins (LDL, HDL, VLDL), total cholesterol, and triglycerides at Day 28; and change from Baseline in lipid metabolism (LBP), 16S rDNA and stool sample for alpha-1-antitrypsin and microbiome/metabolome analysis; and (4) measurement of health-related quality of life (EQ-5D-5L),

We claim:

1. A method of treating an acute liver disease comprising administering to a patient in need thereof an effective amount of Compound 1: 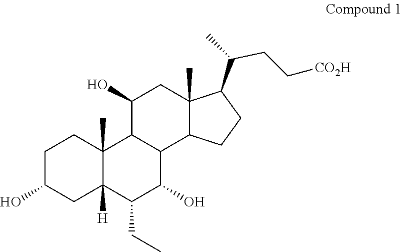 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]
or a pharmaceutically acceptable salt or amino acid conjugate thereof.

2. The method of claim 1, wherein the liver disease is alcoholic hepatitis (AH).

3. The method of claim 1, wherein the liver disease is severe alcoholic hepatitis (sAH).

4. The method of claim 1, wherein the liver disease is acute-on-chronic liver failure (ACLF).

5. The method of any one of claims 1-4, wherein the patient exhibits a Maddrey discriminant function (MDF) score of ≥32 prior to treatment with Compound 1 or a pharmaceutically acceptable salt or amino acid conjugate thereof.

6. The method of any one of claims 1-4, wherein the patient exhibits a model for end-stage liver disease (MELD or MELD-Na) score of ≥18, or greater than 21, or from 21 30 prior to treatment with Compound 1 or a pharmaceutically acceptable salt or amino acid conjugate thereof.

7. The method of claim 6, wherein the patient exhibits a MDF score of 32 to 60.

8. The method of claim 5, wherein the patient exhibits a MELD score of 18 to 30, or 21-30, or MELD-Na score of 18 to 25, or 21-30.

9. The method of any one of claims 5-8, wherein the method reduces the MELD or MELD-Na score of the patient by at least 3 and/or reduces the MDF score of the patient by at least 3 by day 28 or day 90 of treatment.

10. The method of any one of claims 1-9, wherein Compound 1 or a pharmaceutically acceptable salt or amino acid conjugate thereof is administered daily at a dose of about 1.0 mg to about 300 mg.

11. The method of any one of claims 1-10, wherein Compound 1 or a pharmaceutically acceptable salt or amino acid conjugate thereof is administered daily at a dose of about 2.5 mg to about 300 mg.

12. The method of any one of claims 1-11, wherein Compound 1 is administered daily at a dose of from about 5 mg to about 100 mg; or from about 5 mg to about 120 mg.

13. The method of any one of claims 1-12, wherein Compound 1 is administered daily at a dose of from about 5 mg to about 50 mg.

14. The method of any one of claims 1-13, wherein Compound 1 is administered daily at a dose of from about 5 mg to about 25 mg.

15. The method of any one of claims 1-14, wherein Compound 1 is administered daily at a dose of from about 5 mg to about 10 mg.

16. The method of any one of claims 1-10, wherein Compound 1 or a pharmaceutically acceptable salt or amino acid conjugate thereof is administered daily at a dose of about 5 mg to about 150 mg.

17. The method of any one of claims 1-10, wherein Compound 1 or a pharmaceutically acceptable salt or amino acid conjugate thereof is administered daily at a dose of about 10 mg to about 100 mg; or from about 5 mg to about 120 mg.

18. The method of any one of claims 1-10, wherein Compound 1 or a pharmaceutically acceptable salt or amino acid conjugate thereof is administered daily at a dose of about 50 mg to about 200 mg.

19. The method of any one of claims 1-10, wherein Compound 1 is administered daily at a dose of about 2.5 mg, about 5 mg, about 10 mg, about 15 mg, about 20 mg, about 25 mg, about 35 mg, about 45 mg, about 50 mg, about 60 mg, about 75 mg, about 85 mg, about 100 mg, about 115 mg, about 120 mg, about 125 mg, about 140 mg, about 150 mg, about 165 mg, about 175 mg, about 190 mg, or about 200 mg.

20. The method of any one of claims 1-9, wherein Compound 1 or a pharmaceutically acceptable salt or amino acid conjugate thereof is administered in single or multiple doses sufficient to achieve a total daily dose of about 1.0 mg to about 300 mg.

21. The method of any one of claims 1-20, further comprising monitoring blood levels of a biomarker in the patient after administration of Compound 1 or a pharmaceutically acceptable salt or amino acid conjugate thereof, wherein the biomarker is selected from C4, FGF-19, and endogenous bile acids.

22. The method of any one of claims 1-21, wherein the patient has a chronic liver disease in addition to sAH or ACLF.

23. The method of claim 22, wherein the patient has alcoholic liver disease (ALD).

24. The method of any one of claims 1-23, wherein the patient exhibits one or more of the following symptoms: (1) ongoing alcohol consumption of more than 40 g of alcohol per day if the patient is a woman and more than 60 g of alcohol per day if the patient is a man for 6 months or more, with less than 60 days of abstinence before the onset of jaundice; (2) the presence of elevated liver enzymes (aspartate aminotransferase [AST] and alanine aminotransferase [ALT] concentrations≥50 IU/L (or U/L) but ≤400 IU/L (or U/L) and an AST/ALT ratio of ≥1:5); and (3) worsening jaundice, with bilirubin concentrations greater than 3 mg/dL.

25. The method of any one of claims 1-24, wherein the patient exhibits alcohol-associated cirrhosis, liver fibrosis, steatosis, steatohepatitis, alcoholic hepatitis, or intestinal dysbiosis.

26. The method of any one of claims 1-25, wherein the patient has previously been diagnosed with sAH and has suffered at least one relapse of sAH.

27. The method of any one of claims 1-26, wherein the method improves 30-day survival of the patient versus an otherwise similar patient who was not administered Compound 1 or a pharmaceutically acceptable salt or amino acid conjugate thereof.

28. The method of any one of claims 1-27, wherein the method improves 60-day survival of the patient versus an otherwise similar patient who was not administered Compound 1 or a pharmaceutically acceptable salt or amino acid conjugate thereof.

29. The method of any one of claims 1-28, wherein the method improves 90-day survival of the patient versus an otherwise similar patient who was not administered Compound 1 or a pharmaceutically acceptable salt or amino acid conjugate thereof.

30. The method of any one of claims 27-29, wherein the method improves survival by about 10% to about 40%.

31. The method of any one of claims 27-29, wherein the method improves survival by about 10% to about 30%.

32. The method of any one of claims 27-29, wherein the method improves survival by about 10% to about 20%.

33. The method of any one of claims 1-32, wherein the method provides a reduction in health care utilization.

34. The method of claim 33, wherein the method provides a reduction in liver-related events in patients with sAH.

35. The method of claim 33 or 34, wherein the method provides a decrease in the patient's health care utilization by at least 10% for 60 days.

36. The method of any one of claims 33-35, wherein the method reduces one or more of hospital re-admission within 30-days, 60 days or 90 days after discharge, number of hospitalizations, length of stay, emergency room visits, intensive care unit (ICU) days, infectious complications selected from sepsis, pneumonia, urinary tract infection (UTI), cellulitis, spontaneous and bacterial peritonitis (SBP), and major medical procedures.

37. The method of any one of claims 1-36, further comprising co-administering to the patient an effective amount of anti-inflammatory agent, an antibiotic, an antioxidant, a probiotic, a fecal transplant, Zn-supplement or a combination of any of those agents.

38. The method of any one of claims 1-36, further comprising co-administering to the patient an effective amount of pentoxifylline, Augmentin, bovine colostrum, corticosteroid (e.g., prednisolone or methylprednisolone), an ELAD agent, canakinumab, a TNF inhibitor, IL-22, N-acetylcysteine, metadoxine, IgG anti-LPS, a probiotic, a fecal transplant, Zn-supplement, or a combination of any of those agents.

39. The method of any one of claims 1-38, wherein the patient does not exhibit one or more of: AST or ALT>400 U/L, MDF>60 prior to treatment, MELD score>25 or >30 or MELD-Na>25 or >30 prior to treatment, other causes of liver disease selected from chronic hepatitis B (hepatitis B surface antigen [HBsAg] positive), chronic hepatitis C (HCV RNA positive), acetaminophen hepatotoxicity, biliary obstruction, and autoimmune liver disease; concomitant of previous history of hepatocellular carcinoma (HCC), a history of liver transplantation, untreated sepsis, known positivity for human immunodeficiency virus infection, uncontrolled gastrointestinal (GI) bleeding or controlled GI bleeding within 7 days of beginning treatment that was associated with shock or required transfusion of more than 3 units of blood, acute kidney injury defined as a serum creatinine>133 μmol/L (>1.5 mg/dL) or the requirement for renal replacement therapy, portal vein thrombosis, acute pancreatitis, or severe associated disease selected from cardiac failure, acute myocardial infarction, severe cardiac arrhythmias, severe pulmonary disease, and neurologic disease.

40. The method of any one of claims 1-39, wherein the method provides at least a 15% reduction in mortality at 90 days compared to standard of care (SOC).

41. The method of any one of claims 1-40, wherein the method provides a reduction in hospitalization by at least 15% and/or a reduction in progression to transplant by at least 15%.

42. The method of any one of claims 1-41, wherein Compound 1 or a pharmaceutically acceptable salt or amino acid conjugate thereof is administered to the patient for about 30 to about 90 days.

43. The method of any one of claims 1-42, wherein Compound 1 or a pharmaceutically acceptable salt or amino acid conjugate thereof is administered orally.

44. Then method of claim 6, wherein Compound 1 or a pharmaceutically acceptable salt or amino acid conjugate thereof is administered in single or multiple doses sufficient to achieve a total daily dose of about:
1.0 mg to about 300 mg; or
about 2.5 mg to about 300 mg; or
about 5 mg to about 100 mg; or
about 5 mg to about 50 mg; or
about 5 mg to about 25 mg; or
about 5 mg to about 10 mg; or
about 5 mg to about 150 mg; or
about 5 mg to about 120 mg;
about 10 mg to about 100 mg; or
about 50 mg to about 200 mg; or
about 2.5 mg, about 5 mg, about 10 mg, about 15 mg, about 20 mg, about 25 mg, about 35 mg, about 45 mg, about 50 mg, about 60 mg, about 75 mg, about 85 mg, about 100 mg, about 115 mg, about 120 mg, 125 mg, about 140 mg, about 150 mg, about 165 mg, about 175 mg, about 190 mg, or about 200 mg.

45. The method of any one of claims 1-9 wherein the patient exhibits an AST of ≥50 U/L (or IU/L) prior to treatment with Compound 1 or a pharmaceutically acceptable salt or amino acid conjugate thereof.

46. The method of any one of claims 1-9 wherein the patient exhibits an AST/ALT ratio of ≥1.5 prior to treatment with Compound 1 or a pharmaceutically acceptable salt or amino acid conjugate thereof.

47. The method of any one of claims 1-9 wherein the patient exhibits prior to treatment with Compound 1 or a pharmaceutically acceptable salt or amino acid conjugate thereof, one or more of:
$$an AST of \geq 50 U / L ; \\ an AST / ALT ratio of \geq 1.5 ;$$
a Maddrey discriminant function (MDF) score of ≥32; and
a model for end-stage liver disease (MELD or MELD-Na) score of ≥18, or ≥20 or ≥21, for example from 21-30.

48. A method of determining candidacy of a patient for therapeutic treatment for sAH, comprising determining the patient's MELD-Na score.

49. The method of claim 48, further comprising determining the patient's MDF score.

50. The method of claim 49, wherein the patient's MELD-Na score is ≥18.

51. The method of claim 48, wherein the patient's MELD-Na score is ≥18.

52. The method of claim 49, wherein the patient's MDF score is ≥32, for example from 32 to 60 inclusive.

53. A method for treating sAH comprising:
selecting a patient having a MELD-Na score≥18; or a MELD score of 21-30, and
administering a therapeutic treatment for sAH.

54. The method of claim 53, wherein the patient also has a MDF score from 32 to 60 inclusive.

55. The method of claim 53 or 54, wherein the therapeutic treatment comprises administering a pharmacologic agent to the patient, and/or a liver transplant.
